SEQUENCE LISTING (27 sequences)

SEQ ID NO: 1 - protein, 88 aa, Artificial Sequence
  Synthetic GP41-1 IN (WT)
CLDLKTQVQTPQGMKEISNIQVGDLVLSNTGYNEVLNVFPKSKKKSYKITLEDGKEIICSEEHLFPTQTGEMNISGGLKEGMCLYVKE

SEQ ID NO: 2 - protein, 37 aa, Artificial Sequence
  Synthetic GP41-1 IC (WT)
MMLKKILKIEELDERELIDIEVSGNHLFYANDILTHN

SEQ ID NO: 3 - protein, 100 aa, Artificial Sequence
  Synthetic GP41-1 IN (C25)
CLDLKTQVQTPQGMKEISNIQVGDLVLSNTGYNEVLNVFPKSKKKSYKITLEDGKEIICSEEHLFPTQTGEMNISGGLKEGMCLYVKEMMLKKILKIEEL

SEQ ID NO: 4 - protein, 25 aa, Artificial Sequence
  Synthetic GP41-1 IC (C25)
DERELIDIEVSGNHLFYANDILTHN

SEQ ID NO: 5 - protein, 98 aa, Artificial Sequence
  Synthetic IN (C27)
CLDLKTQVQTPQGMKEISNIQVGDLVLSNTGYNEVLNVFPKSKKKSYKITLEDGKEIICSEEHLFPTQTGEMNISGGLKEGMCLYVKEMMLKKILKIE

SEQ ID NO: 6 - protein, 27 aa, Artificial Sequence
  Synthetic IC (C27)
ELDERELIDIEVSGNHLFYANDILTHN

SEQ ID NO: 7 - protein, 99 aa, Artificial Sequence
  Synthetic IN (C26)
CLDLKTQVQTPQGMKEISNIQVGDLVLSNTGYNEVLNVFPKSKKKSYKITLEDGKEIICSEEHLFPTQTGEMNISGGLKEGMCLYVKEMMLKKILKIEE

SEQ ID NO: 8 - protein, 26 aa, Artificial Sequence
  Synthetic IC (C26)
LDERELIDIEVSGNHLFYANDILTHN

SEQ ID NO: 9 - protein, 101 aa, Artificial Sequence
  Synthetic IN (C24)
CLDLKTQVQTPQGMKEISNIQVGDLVLSNTGYNEVLNVFPKSKKKSYKITLEDGKEIICSEEHLFPTQTGEMNISGGLKEGMCLYVKEMMLKKILKIEELD

SEQ ID NO: 10 - protein, 24 aa, Artificial Sequence
  Synthetic IC (C24)
ERELIDIEVSGNHLFYANDILTHN

SEQ ID NO: 11 - protein, 102 aa, Artificial Sequence
  Synthetic IN (C23)
CLDLKTQVQTPQGMKEISNIQVGDLVLSNTGYNEVLNVFPKSKKKSYKITLEDGKEIICSEEHLFPTQTGEMNISGGLKEGMCLYVKEMMLKKILKIEELDE

SEQ ID NO: 12 - protein, 23 aa, Artificial Sequence
  Synthetic IC (C23)
RELIDIEVSGNHLFYANDILTHN

SEQ ID NO: 13 - protein, 103 aa, Artificial Sequence
  Synthetic IN (C22)
CLDLKTQVQTPQGMKEISNIQVGDLVLSNTGYNEVLNVFPKSKKKSYKITLEDGKEIICSEEHLFPTQTGEMNISGGLKEGMCLYVKEMMLKKILKIEELDER

SEQ ID NO: 14 - protein, 22 aa, Artificial Sequence
  Synthetic IC (C22)
ELIDIEVSGNHLFYANDILTHN

SEQ ID NO: 15 - protein, 108 aa, Artificial Sequence
  Synthetic IN (C17)
CLDLKTQVQTPQGMKEISNIQVGDLVLSNTGYNEVLNVFPKSKKKSYKITLEDGKEIICSEEHLFPTQTGEMNISGGLKEGMCLYVKEIMLKKILKIEELDERELIDI

SEQ ID NO: 16 - protein, 17 aa, Artificial Sequence
  Synthetic IC (C17)
EVSGNHLFYANDILTHN

SEQ ID NO: 17 - protein, 112 aa, Artificial Sequence
  Synthetic IN (C13)
CLDLKTQVQTPQGMKEISNIQVGDLVLSNTGYNEVLNVFPKSKKKSYKITLEDGKEIICSEEHLFPTQTGEMNISGGLKEGMCLYVKEMMLKKILKIEELDERELIDIEVSG

SEQ ID NO: 18 - protein, 13 aa, Artificial Sequence
  Synthetic IC (C13)
NHLFYANDILTHN

SEQ ID NO: 19 - protein, 262 aa, Artificial Sequence
  Synthetic FRB (IN)
MRVAILWHEMWHEGLEEASRLYFGERNVKGMFEVLEPLHAMMERGPQTLKETSFNQAYGRDLMEAQEWCRKYMKSGNVKDLTQAWDLYYHVFRRISYPAFLYKVVDSRGGGGSGGGGSYPYDVPDYAGGSYPYDVPDYAGGGSGKPIPNPLLGLDSTTRSGYCLDLKTQVQTPQGMKEISNIQVGDLVLSNTGYNEVLNVFPKSKKKSYKITLEDGKEIICSEEHLFPTQTGEMNISGGLKEGMCLYVKEMMLKKILKIEEL

SEQ ID NO: 20 - protein, 274 aa, Artificial Sequence
  Synthetic FRB (NIN)
MYPYDVPDYAGGSYPYDVPDYAGGGSGKPIPNPLLGLDSTTRSGYCLDLKTQVQTPQGMKEISNIQVGDLVLSNTGYNEVLNVFPKSKKKSYKITLEDGKEIICSEEHLFPTQTGEMNISGGLKEGMCLYVKEMMLKKILKIEELGGSGGGGSGGGGSKLTSLYKKAGLMRVAILWHEMWHEGLEEASRLYFGERNVKGMFEVLEPLHAMMERGPQTLKETSFNQAYGRDLMEAQEWCRKYMKSGNVKDLTQAWDLYYHVFRRISYPAFLYKVV

SEQ ID NO: 21 - protein, 204 aa, Artificial Sequence
  Synthetic FKBP1A (IC)
MDERELIDIEVSGNHLFYANDILTHNSSSDVGTDYKDHDGDYKDHDIDYKDDDDKGGGGSGGGGSEQKLISEEDLLTSLYKKAGSTMGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESDPAFLYKVV

SEQ ID NO: 22 - protein, 199 aa, Artificial Sequence
  Synthetic FKBP1A (CIC)
MGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESDPAFLYKVVDSRGGGGSGGGGSGMDERELIDIEVSGNHLFYANDILTHNSSSDVGTDYKDHDGDYKDHDIDYKDDDDKGSEQKLISEEDL

SEQ ID NO: 23 - protein, 440 aa, Artificial Sequence
  Synthetic FKBP1A (CIC-GFP)
MGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESDPAFLYKVVDSRGGGGSGGGGSGMDERELIDIEVSGNHLFYANDILTHNSSSDVGTDYKDHDGDYKDHDIDYKDDDDKGSEQKLISEEDLSRMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 24 - protein, 340 aa, Artificial Sequence
  Synthetic Product of FRB (IN) + FKBP1A (IC)
MRVAILWHEMWHEGLEEASRLYFGERNVKGMFEVLEPLHAMMERGPQTLKETSFNQAYGRDLMEAQEWCRKYMKSGNVKDLTQAWDLYYHVFRRISYPAFLYKVVDSRGGGGSGGGGSYPYDVPDYAGGSYPYDVPDYAGGGSGKPIPNPLLGLDSTTRSGYSSSDVGTDYKDHDGDYKDHDIDYKDDDDKGGGGSGGGGSEQKLISEEDLLTSLYKKAGSTMGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESDPAFLYKVV

SEQ ID NO: 25 - protein, 203 aa, Artificial Sequence
  Synthetic Product of FRB (IN) + FKBP1A (CIC)
MRVAILWHEMWHEGLEEASRLYFGERNVKGMFEVLEPLHAMMERGPQTLKETSFNQAYGRDLMEAQEWCRKYMKSGNVKDLTQAWDLYYHVFRRISYPAFLYKVVDSRGGGGSGGGGSYPYDVPDYAGGSYPYDVPDYAGGGSGKPIPNPLLGLDSTTRSGYSSSDVGTDYKDHDGDYKDHDIDYKDDDDKGSEQKLISEEDL

SEQ ID NO: 26 - protein, 223 aa, Artificial Sequence
  Synthetic Product of FRB (NIN) + FKBP1A (IC)
MYPYDVPDYAGGSYPYDVPDYAGGGSGKPIPNPLLGLDSTTRSGYSSSDVGTDYKDHDGDYKDHDIDYKDDDDKGGGGSGGGGSEQKLISEEDLLTSLYKKAGSTMGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESDPAFLYKVV

SEQ ID NO: 27 - protein, 327 aa, Artificial Sequence
  Synthetic Product of FRB (NIN) + FKBP1A (CIC-GFP)
MYPYDVPDYAGGSYPYDVPDYAGGGSGKPIPNPLLGLDSTTRSGYSSSDVGTDYKDHDGDYKDHDIDYKDDDDKGSEQKLISEEDLSRMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

(19) United States
(12) Patent Application Publication
(10) Pub. No.: US 2022/0229068 A1
(43) Pub. Date: Jul. 21, 2022

(54) DETECTION OF PROTEIN TO PROTEIN INTERACTIONS

(71) Applicant: THE GOVERNING COUNCIL OF THE UNIVERSITY OF TORONTO

(72) Inventors: Zhong YAO, Toronto (CA); Igor STAGLJAR, Toronto (CA)

(73) Assignee: THE GOVERNING COUNCIL OF THE UNIVERSITY OF TORONTO, Toronto (CA)

(21) Appl. No.: 17/618,252

(22) PCT Filed: Jun. 30, 2020
(86) PCT No.: PCT/CA2020/050914

§ 371 Date: Dec. 10, 2021

(30) Foreign Application Priority Data
Jul. 1, 2019 (GB) 1909491.1

(51) Int. Cl. G01N 33/68; C12N 15/10

(52) U.S. Cl. CPC G01N 33/6845; C12N 15/1055

(57) ABSTRACT

A system and method for detecting interactions between a first protein or fragment thereof (bait protein) and a second protein or fragment thereof (prey protein) comprising: (a) a bait construct comprising the bait protein, a first epitope tag and an intein N-terminal fragment (IN); and (b) a prey construct comprising the prey protein, a second epitope tag, and an intein C-terminal fragment (IC).

### FIELD OF THE INVENTION

The present disclosure is relates to novel reagents for methods for detecting protein-protein interactions with an in vivo genetic system.

### BACKGROUND OF THE INVENTION

Proteins never work alone, and protein-protein interactions (PPIs) compose many fundamental steps in most biological processes [1]. Deregulation of PPIs can be a key driving force in disease development. Thus, PPI detection and analysis are essential for understanding molecular mechanisms of biological processes, elucidating mechanistic details of disease occurrence and progression, as well as for developing new treatments and novel diagnostics.

Numerous methods, including high-throughput (HTP) techniques, have been developed to detect PPIs [1, 2]. They have advanced biological research and dramatically changed our views on cell function. However, based on the underpinning principles of their design, these methods present bias for certain sets of PPIs and are accompanied by limitations [3]. For example, stable PPIs are often easily monitored by various methods, but transient and weak PPIs, which play essential regulatory roles, are far more difficult to detect. PPIs occurring in some special cellular locations can also only be examined with specific methods. Additionally, some approaches reconstitute PPIs in model organisms such as yeast [4], which may not accurately represent native physiological environments, while methods based on affinity purification are usually biased by overrepresentation of abundant proteins and underrepresentation of weak PPIs that are easily lost during purification. Many methods also suffer from low quantifiability or throughput. There is therefore no universal method that works for every PPI.

An intein is a protein fragment possessing enzymatic activity which allows it to excise itself from its parental peptide while ligating (via formation of a peptide bond) the protein regions flanking it (referred to as the N-terminal extein (EN) and the C-terminal extein (EC)) into a new intact peptide through a process called protein splicing (FIG. 1H) [5, 6]. Inteins usually are small or can be reduced to a small domain close to 100 amino acids. Their function does not require any cofactors or energy source and usually can work across relatively broad environmental conditions. Interestingly, an intein can be split into two parts, either naturally or artificially, without compromising its activity and thereby allowing protein trans-splicing [7], thus making such split inteins attractive tools in biotechnological fields [8]. The same features also allowed us to set up the SIMPL system.

### SUMMARY OF THE INVENTION

In one embodiment, the present disclosure describes an artificial split intein comprising a C-terminus fragment (IC) that includes amino acid residues at positions 13 to 37 of wild type IC of GP41-1, and a N-terminus fragment (IN) that includes amino acid residues at positions 1 to 88 of the wild type IN of GP41-1 fused to amino acid residues 1 to 12 of wild type IC of GP41-1 (C25 GP41-1 split intein), or (ii) the IC includes amino acids at positions 14 to 37 of wild type IC of GP41-1 and amino acid residues at positons 1 to 88 of wild type IN of GP41-1 fused to amino acid residues at positions 1 to 13 of wild type IC of GP41-1 (C24 GP41-1 split intein), or (iii) the IC includes amino acids at positions 15 to 37 of wild type IC of GP41-1 and amino acid residues at positons 1 to 88 of wild type IN of GP41-1 fused to amino acid residues at positions 1 to 14 of wild type IC of GP41-1 (C23 GP41-1 split intein).

In one embodiment, this disclosure describes a system for detecting interactions between a first protein or fragment thereof (bait protein) and a second protein or fragment thereof (prey protein) comprising: (a) a bait construct comprising the bait protein, a first epitope tag and an intein N-terminal fragment (IN); and (b) a prey construct comprising the prey protein, a second epitope tag, and an intein C-terminal fragment (IC).

In one embodiment of the system of the present disclosure, the bait protein is fused at its C-terminus to the IN through the first epitope tag, and wherein the prey protein is fused at its N-terminus to the IC through the second epitope tag.

In another embodiment of the system of the present disclosure, the IN is fused to the N-terminal end of the bait protein while keeping the first epitope tag upstream (NIN), and the prey protein is fused at its N-terminus to the IC through the second epitope tag.

In another embodiment of the system of the present disclosure, the bait protein is fused at its C-terminus to the IN through the first epitope tag, and wherein the IC is fused to the C-terminal end of the prey while keeping the second epitope tag downstream (CIC).

In another embodiment of the system of the present disclosure, the bait construct further comprises a third epitope tag in tandem with the first tag.

In another embodiment of the system of the present disclosure, the first epitope tag, the second epitope tag and the third epitope tag include FLAG, V5-tag, Myc-tag, hemagglutinin (HA)-tag, Spot-tag and NE-tag.

In another embodiment of the system of the present disclosure, the intein is wild type GP41-1.

In another embodiment of the system of the present disclosure, the IC includes amino acid residues at positions 1 to 37 of the IC of GP41-1 (WT IC), and IN includes amino acid residues at positions 1 to 88 of the IN of GP41-1 (WT IN) (C37 GP41-1 split intein).

In another embodiment of the system of the present disclosure, (i) the IC includes amino acid residues at positions 13 to 37 of the IC of GP41-1 (WT IC) and the IN includes amino acid residues at positions 1 to 88 of the IN of GP41-1 (WT IN) and amino acid residues at positions 1 to 12 of WT IC (C25 GP41-1 split intein), or (ii) the IC includes amino acids at positions 14 to 37 of WT IC and amino acid residues at positons 1 to 88 of WT IN and amino acid residues at positions 1 to 13 of WT IC (C24 GP41-1 split intein), or (iii) the IC includes amino acids at positions 15 to 37 of WT IC and amino acid residues at positons 1 to 88 of WT IN and amino acid residues at positions 1 to 14 of WT IC (C24 GP41-1 split intein).

In another embodiment of the system of the present disclosure, the bait protein is a soluble or membrane protein or fragment thereof.

In another embodiment of the system of the present disclosure, the prey protein is a soluble or membrane protein or fragment thereof.

In another embodiment, the present disclosure provides for a method for detecting the interaction between a first protein or part thereof (bait protein) and a second protein or part thereof (prey protein). In one embodiment, the method includes: (a) providing a bait construct comprising the bait protein, a first epitope tag and an intein N-terminal fragment (IN); (b) providing a prey construct comprising the prey protein, a second epitope tag, and an intein C-terminal fragment (IC), wherein an association of the bait protein and the prey protein results in the IN and IC reconstituting into a functional intein molecule, which then catalyzes its excision and formation of an intact protein which includes the first epitope tag and the second epitope tag; (c) incubating the bait construct and the prey construct under conditions that allow the formation of the intact protein to form an incubate; and (d) adding to the incubate an antibody or antibodies that recognize at least one or both of the first epitope and the second epitope to detect the formation of the intact protein, detection of the intact protein being indicative that the first protein or part thereof and the second protein or part thereof interact.

In another embodiment of the method for detecting the interaction between the bait protein and the prey protein, the method further includes measuring an expression output of the detected intact protein as a measure of an amount of interaction between the first and the second proteins to quantitatively measure strength and affinity between the bait protein and the prey protein.

In another embodiment of the method for detecting the interaction between the bait protein and the prey protein, the bait construct and the prey construct are expressed in a host cell.

In another embodiment of the method for detecting the interaction between the bait protein and the prey protein, the method includes: (i) introducing into the host cell as part of a bait vector, a first gene under the control of a promoter, said first gene coding inter alia for the bait protein which gene is attached to the DNA-sequence of a first module encoding inter alia the first epitope tag and the IN; and (ii) introducing into the host cell, as part of a prey vector, a second gene under the control of a promoter, the second gene coding inter alia for the prey protein which gene is attached to the DNA sequence of a second module encoding inter alia the second epitope tag and the IC.

In another embodiment of the method for detecting the interaction between the bait protein and the prey protein, the bait vector is maintained episomally in the host mammalian cell or is integrated into the genome of the host mammalian cell.

In another embodiment of the method for detecting the interaction between the bait protein and the prey protein, the prey vector is maintained episomally in the host mammalian cell or is integrated into the genome of the host mammalian cell.

In another embodiment of the method for detecting the interaction between the bait protein and the prey protein, the bait construct further comprises a third epitope tag in tandem with the first epitope tag, and wherein the method further comprises performing another incubation of the incubate on a substrate coated with an antibody against either the first epitope tag or the third epitope tag.

In another embodiment of the method for detecting the interaction between the bait protein and the prey protein, the detection is performed as an ELISA assay.

In another embodiment, the present disclosure provides for a method of identifying a potentially pharmaceutically active agent. In one embodiment, the method includes: (a) providing a host cell; (b) expressing in the host cell a bait construct comprising the bait protein a first epitope tag and an intein N-terminal fragment (IN); (c) expressing in the host cell a prey construct comprising the prey protein, a second epitope tag, and an intein C-terminal fragment (IC), the bait protein and the prey protein being selected such that they interact when expressed in the host cell, wherein the interaction of the bait protein and the prey protein results in the IN and IC reconstituting into a functional intein molecule, which then catalyzes its excision and formation of an intact protein which includes the first epitope tag and the second epitope tag; (d) incubating the host cell in presence of an agent under conditions that allow for the formation of the intact protein to form an incubate; and (e) adding to the incubate an antibody or antibodies that recognize at least one or both of the first epitope and the second epitope to detect the formation of the intact protein, wherein an absence of detection of the intact protein is indicative of the agent being potentially pharmaceutically active.

In another embodiment, the present disclosure provides for a method for providing a compound that can interfere with protein/protein interaction, the method including: (a) providing a host cell having the bait vector described and the prey vector described in the fourth embodiment of the system of the present disclosure, the bait protein and the prey protein being selected such that they interact when expressed; (b) incubating the host cell in the presence and absence of the compound(s) to be tested; (c) measuring the difference in expression between the incubation containing the compound(s) to be tested and the incubation free of the compound(s) to be tested; and optionally (d) purifying or synthesizing the compound that can interfere with protein-protein interaction.

In one embodiment according to any of the methods of the present disclosure, the IC includes amino acid residues at positions 1 to 37 of the IC of GP41-1 (WT IC), and IN includes amino acid residues at positions 1 to 88 of the IN of GP41-1 (WT IN) (C37 GP41-1 split intein).

In one embodiment according to any of the methods of the present disclosure, (i) the IC includes amino acid residues at positions 13 to 37 of the IC of GP41-1 (WT IC) and the IN includes amino acid residues at positions 1 to 88 of the IN of GP41-1 (WT IN) and amino acid residues at positions 1 to 12 of WT IC (C25 GP41-1 split intein), or (ii) the IC includes amino acids at positions 14 to 37 of WT IC and amino acid residues at positons 1 to 88 of WT IN and amino acid residues at positions 1 to 13 of WT IC (C24 GP41-1 split intein), or (iii) the IC includes amino acids at positions 15 to 37 of WT IC and amino acid residues at positons 1 to 88 of WT IN and amino acid residues at positions 1 to 14 of WT IC (C24 GP41-1 split intein).

In another embodiment, the present disclosure is a split intein comprising a C-terminus fragment (IC) that includes amino acid residues at positions 13 to 37 of wild type IC of GP41-1, and a N-terminus fragment (IN) that includes amino acid residues at positions 1 to 88 of the wild type IN of GP41-1 and amino acid residues at positions 1 to 12 of wild type IC (C25 GP41-1 split intein).

In another embodiment, the present disclosure is a split intein comprising a C-terminus fragment (IC) that includes amino acid residues at positions 14 to 37 of wild type IC of GP41-1, and a N-terminus fragment (IN) that includes amino acid residues at positions 1 to 88 of the wild type IN of GP41-1 and amino acid residues at positions 1 to 13 of wild type IC (C24 GP41-1 split intein).

In another embodiment, the present disclosure is a split intein comprising a C-terminus fragment (IC) that includes amino acid residues at positions 15 to 37 of wild type IC of GP41-1, and a N-terminus fragment (IN) that includes amino acid residues at positions 1 to 88 of the wild type IN of GP41-1 and amino acid residues at positions 1 to 14 of wild type IC (C24 GP41-1 split intein).

In one embodiment of any of the methods of the present disclosure, the bait protein is fused at its C-terminus to the IN through the first epitope tag, and wherein the prey protein is fused at its N-terminus to the IC through the second epitope tag.

In another embodiment of any of the methods of the present disclosure, the IN is fused to the N-terminal end of the bait protein while keeping the first epitope tag upstream (NIN), and the prey protein is fused at its N-terminus to the IC through the second epitope tag.

In another embodiment of any of the methods of the present disclosure, the bait protein is fused at its C-terminus to the IN through the first epitope tag, and wherein the IC is fused to the C-terminal end of the prey while keeping the second epitope tag downstream (CIC).

In another embodiment of any of the methods of the present disclosure, the bait construct further comprises a third epitope tag in tandem with the first tag.

In another embodiment of any of the methods of the present disclosure, the first epitope tag, the second epitope tag and the third epitope tag include FLAG, V5-tag, Myc-tag, hemagglutinin (HA)-tag, Spot-tag and NE-tag.

In another embodiment, the present disclosure is a sensor for protein interactions comprising a split intein.

In another embodiment, the present disclosure relates to an isolated peptide comprising SEQ ID NO:3.

In another embodiment, the present disclosure relates to an isolated peptide comprising SEQ ID NO:4.

### BRIEF DESCRIPTION OF THE DRAWINGS

Embodiments will now be described, by way of example only, with reference to the drawings, in which:

FIGS. 1A to 1J: Development of the SIMPL assay. (A) The design of SIMPL for PPI detection. (B) Schematic representation of SIMPL bait and prey constructs and the resplitting of the GP41-1 intein. (C) Examination of SIMPL system with GP41-1 split intein with different splitting sites. DNA constructs coding for FRB-IN and IC-FKBP1A with inteins split at the sites were expressed in HEK 293 cells. After incubation with rapamycin (100 nM) for 2 hrs, the cells were lysed and the lysates subjected to Western blot analysis with α-V5 and α-FLAG antibodies. Both IN and IC constructs with C25 splitting exhibited the best performance and were adopted as the standard sensor intein for the SIMPL platform. (D) Dose response of rapamycin-induced FRB/FKBP1A interaction examined with SIMPL. HEK 293 cells expressing FRB-IN and IC-FKBP1A were treated with the indicated doses of rapamycin for 2 hrs followed by Western blot analysis. The densities of spliced bands (FRB-FKBP1A) were quantified with ImageJ and are presented as bar graphs above the blots. (E-G) Time course of rapamycin-induced FRB/FKBP interaction. HEK 293 (E), HeLa (F) or PC9 (G) cells expressing FRB-IN and IC-FKBP1A were treated with rapamycin (100 nM) for different periods of time as indicated followed by Western blot analysis. (H) Stable cells derived from HEK 293 T-Rex FlpIn with FRB-IN and IC-FKBP1A inserted into the FRT site were treated with the indicated different concentrations of tetracycline for 16 h, followed by treatment with rapamycin (100 nM) for 2 h and then analysis by western blot. HEK 293 cells transiently transfected with FRB-IN and IC-FKBP1A were used as a control (right two lanes). The blot is representative of three independent experiments. (I) Mechanism of protein splicing reaction. “X” stands for “S” or “O”. (J) Schematic representation of wild type IC and wild type IN (C37) and artificial splits C27, C26, C25, C24, C23, C22, C17 and C13.

FIGS. 2A to 2B: Design of alternative formats of SIMPL to expand its capability. (A) The IN/IC formats allow splicing between bait and prey. In the CIC orientation, IC-FLAG is fused to the C-terminus of a prey protein (Prey-IC-FLAG). Its combination with IN bait (Bait-V5-IN) leads to the transfer of FLAG tag to the bait generating Bait-V5-FLAG. In the NIN orientation, V5-IN is fused to the N-terminus of a bait (V5-IN-Bait). Its use with IC prey (IC-FLAG-Prey) causes the transfer of V5 tag to the prey thus generating V5-FLAG-Prey. The CIC-GFP construct (Prey-IC-FLAG-GFP) is created to allow the detection of NIN/CICGFP combination, which produces a V5-FLAG-GFP peptide. (B) The performance of different SIMPL formats were experimentally assessed using rapamycin-induced FRB/FKBP1A interaction in which the corresponding bait and prey constructs were transiently transfected. Bands of spliced products are highlighted with triangles and parental proteins are highlighted with asterisks. The densities of spliced bands (FRB-FKBP1A) were quantified with ImageJ and are presented as bar graphs above the blots. The blot is representative of three independent experiments.

FIGS. 3A to 3K: Setting up the SIMPL ELISA platform and its evaluation with reference PPIs. (A) An extra HA tag is introduced into the bait construct. The spliced proteins are captured by immobilized α-FLAG antibody and measured with α-HA antibody conjugated with HRP. All four SIMPL formats described in FIG. 2 are compatible with ELISA. (B) The bait proteins can be measured similarly by ELISA using immobilized α-V5 antibody and HRP-conjugated α-HA antibody probe. (C) The ELISA platform was assessed with the rapamycin-induced FRB/FKBP1A interaction. HEK 293 cells expressing FRB and FKBP1A in different formats were treated with different doses of rapamycin as indicated for 30 min followed by lysis and ELISA analysis. The experiment was performed with four technical replicates and each replicate is presented as a single dot. (D) Benchmarking analysis of the overall performance of SIMPL ELISA platform. Eighty-eight PPIs well documented in literature were chosen as positive reference set (PRS). Eighty-eight pairs of bait/prey combinations with the least possibility of interaction were selected from the bait and preys of the PRS to form the random reference set (RRS). Both sets were then screened using SIMPL ELISA analysis in both IN/IC and IN/CIC formats. The spliced signal was normalized to bait expression. Receiver operating characteristic (ROC) analysis was performed as presented. Data shown here are a representative result of three experiments. (E) Performance of the SIMPL assay in terms of sensitivity (true-positive rate) and false-positive rate (1-specificity). The threshold values for positive detection were determined from ROC analysis as in d. Results are averages of three independent experiments showing mean recovery rate±SEM. (F) Comparison of SIMPL detection of individual PPIs in the PRS to results from seven different PPI methods obtained from the literature (Braun et al, 2009; Lievens et al, 2014; Trepte et al, 2018). Performance of ELISA coupled SIMPL evaluated with reference PPIs. (G) Plot of bait expression for data in FIG. 3d. Samples with mock transfection or unrelated vectors are highlighted with black. (H) Heatmap of splicing signal (normalized by bait signal) with prey in either IC format or CIC format. PRS pairs are symbolized with black rectangles and the RRS pairs are symbolized with white rectangles in the upper row. (I, J) Comparison of SIMPL ELISA and Western blot readouts. PPI pairs from the PRS, either in IN/IC (I) or IN/CIC format (J), with high signal or low signal obtained in ELISA assay, were re-tested by Western blot. Bands of parental proteins are highlighted with asterisks. Bands of spliced proteins or their predicted positions are labelled with triangles. The densities of spliced bands were quantified and are presented as bar graphs above the blots. OL indicates overlap of a spliced band and its parental bands, which prevents accurate measurement of the band. In each panel, electrophoreses were performed on two separate gels but the samples were transferred and probed on the same nitrocellulose membrane. (K) Comparison of the performance of IC and CIC prey formats. The SIMPL readings from the PRS analysis (FIG. 3D) are plotted as IC vs CIC. Prey proteins with signal peptides are highlighted in red and listed at the right side of the scatter plot.

FIG. 4. Detection of physiological PPIs and their inhibition with SIMPL. (A) EGFR/SHC1 interaction. EGFR WT, inactive (D855A), or constitutively active (L858R) mutants in IN format were co-expressed with SHC1 (IC) in HEK 293 cells. Their interactions were analyzed with western analysis. (B) KRAS/RAF interaction. RAF1-IN and IC-KRAS (WT, inactive 517N mutant, or active G12D or Q61H mutant) were transiently expressed in HEK 293 cells followed by western analysis. (C) Stable cells derived from HEK 293 T-Rex FlpIn with EGFR (IN) and SHC1 (IC) inserted into the FRT site were treated with the indicated different concentrations of tetracycline for 6 h, followed by treatment with EGF (100 ng/ml) for 2 min and then analysis by western blot. s.e. short exposure, I.e. long exposure. Each blot in A-C is representative of three independent experiments. (D) SIMPL analysis of kinase/substrate interactions. The indicated kinase-IN constructs were individually expressed along with their substrates in either IC or CIC format and their interactions were detected by ELISA assay. LSM2 was used as a negative control prey. (E) Mitochondrial PPIs. The selected mitochondrial bait proteins were constructed in IN format. They were then co-expressed with the indicated preys in either IC or CIC format, or in both. The interactions were examined with ELISA. LSM2 was used as a negative control prey. OMM outer mitochondrial membrane, IMS intermembrane space, IMM inner mitochondrial membrane. (F) Retest of PDHA1/PDHB interaction with ELISA-coupled SIMPL. In one sample (the fourth from left), the mitochondrial targeting sequence of PDHA1 was replaced by a nuclear localization sequence. In the last sample (the first from right), WT PDHA1 and PDHB were expressed in separate cells and the cell lysates were mixed for ELISA. The experiments in D-F were performed in triplicates and their mean values are presented as bars with each replicate shown in a single dot. LSM2 was used as a negative control prey. OMM: outer mitochondrial membrane; IMS: intermembrane space; IMM: inner mitochondrial membrane.

FIGS. 5A to 5G. SIMPL for enzymatic/PPI inhibitor identification. (A) Time schedule for studying enzymatic/PPI inhibitors with SIMPL. To avoid splicing before inhibition can occur, an inhibitor has to be administered before protein expression. As the expression is under the control of Tet-on promoter, tetracycline is added to the cells alongside the inhibitor 6 h before assay. (B) Studying an EGFR kinase inhibitor with SIMPL. EGFR kinase inhibitor AG1478 at different indicated doses was incubated with the cells expressing EGFR-IN and IC-SHC1. The spliced EGFR-SHC1 band observed by western blot diminished with increasing AG1478 concentration. The blot is representative of three independent experiments. (C-E) BAX/BCL2 interaction was assayed in different formats as indicated by western analysis. In the case of NIN-BAX/IC-BCL2, immunoprecipitation was performed to resolve the spliced protein from its parental protein since they have similar mobility upon electrophoresis (E). Each blot is representative of three independent experiments. (F) Heatmap of SIMPL ELISA readings of BAX/BCL2 interaction in different formats. Gray color: not tested. LSM3 and LSM2 were used as negative controls for bait and prey respectively. (G) Investigation of the BCL2/BAX PPI inhibitor venetoclax with SIMPL. Cells expressing NIN-BAX and IC-BCL2 (or BCL2L1 as control) were treated with venetoclax (or osimertinib as a negative control) at different concentrations as indicated. The cells were then subjected to ELISA analysis. SIMPL signals were normalized to bait expression. The experiment was performed in triplicates and each replicate is presented as a single dot.

FIGS. 6A-C: Setting up the SIMPL system. (A) Assessment of SIMPL assay with WT or reengineered (C25) split intein GP41-1 using rapamycin-induced FRB/FKBP1A interaction. (B) Three-dimensional crystal structure of GP41-1 split intein modelled by SWISS-MODEL. Crystal coordinate file 6QAZ was retrieved from PDB (Beyer et al, 2019). The natural splitting site at C37 and the resplitting site (C25) used in the SIMPL system are highlighted. (C) Characterizing the identity of spliced protein with immunoprecipitation. Cells expressing FRB-IN, IC-FKBP1A or both were treated with rapamycin or left untreated. The proteins were immunoprecipitated with α-FLAG or α-V5 antibodies and subjected to Western analysis with indicated antibodies. HC: antibody heavy chain; LC: light chain. The blot is representative for three independent experiments.

FIGS. 7A-G: Using SIMPL to study physiological PPIs. (A) The interaction between EGFR (in IN format) and SHC1 were detected with either SHC1 (IC) or SHC1 (CIC) formats. (B) KRAS (in IC format)/RAF1 interaction were detected with both RAF1-IN and NIN-RAF1 formats. Each blot in (a,b) is representative for three independent experiments. (C) EGFR (IN) and SHC1 (IC) were co-transfected into HEK 293 cells. After 16 hours starvation (DMEM supplemented with 0.1% FCS), the cells were stimulated with EGF (100 ng/ml) for indicated periods of time. The cells were subjected to Western blot analysis. The blot is representative for four independent experiments. (D-G) Kinase/substrate interactions were followed by SIMPL. The plasmids coding the indicated kinases in IN format and related substrates in IC format were transfected in HEK 293 Flp-In T-Rex cells. The expression was induced by incubation with tetracycline for 6 hrs in starvation medium. The indicated kinases are either in basal state or activated by related stimulation for 30 mins. Activation of the kinases can be judged by the mobility up-shift of substrate and spliced bands derived from phosphorylation. Each blot in (d-g) is representative for two to three independent experiments.

FIG. 8: Detecting PPIs in C. elegans with SIMPL. (A) Scheme for creating and testing transgenic C. elegans lines for use with the SIMPL ELISA assay. Initially, 15 positive reference set (PRS) PPI pairs were successfully cloned and injected into C. elegans, along with 21 random (RRS) PPI pairs, in both the IN/IC and IN/CIC configurations (72 total lines injected). Potential transgenic lines were screened by western blot for expression of both bait and prey constructs, resulting in 10 total PRS lines and 13 total RRS lines (IC+CIC), representing 7 and 9 distinct PPI pairs, respectively. (B) Performance of the SIMPL ELISA assay in two biological replicates, with the fraction of PPI pairs that are positive at different cutoff values. Cutoffs were set as the ratio of spliced signal to total signal. A biological replicate was reported as the average value of two technical replicates. (C) Visualization of SIMPL ELISA results of the individually tested PPIs. The cutoff value used to determine whether a given PPI was positive or negative was 0.50 (arrow in B), which maximized the number of true-positive and true-negative interactions between both biological replicates. Pairs belonging to the RRS were never positive in both replicates compared to the PRS.

FIG. 9. Western blot screen results of C. elegans lines. Western blot results for the C. elegans SIMPL lines generated. Analysis of each protein pair consists of a blot using the V5 antibody to detect the Prey protein, and the FLAG antibody to detect the Bait protein. Size in kilodaltons is shown for each blot. Red asterisk indicates a non-specific V5 band, while splicing is indicated by a black arrow at the expected product size. The blot is representative for two independent experiments.

FIG. 10. Cell line creation with stable bait and prey expression. A. Plasmid construct used to insert RBD-IN and IC-KRAS into host cell genome. Expression of both genes are under control of CMV-Tet On (TO) promoter. The insert is mediated by Flp-ln recombinase at the FRT site. B. The cells stably incorporated with RBD/KRAS (WT, G12C or G12V) were treated simultaneously with Tetracycline (1 μg/ml) and AMG510 (0.1 μM) for 16 hours. The cell lysates were then analyzed using Western blot analysis. Samples without treatment (Tetracycline or AMG510) were used as controls.

FIG. 11. SIMPL coupled to HTRF assay. A. HEK 293 cells were transfected with FRB-IN and IC-FKBP1A plasmids. The cells were treated with Rapamycin with the indicated concentrations for 2 hours. The cells were then lysed and the lysates were incubated with α-FLAG-Tb and α-HA-d2 antibodies followed by read-out using HTRF compatible fluorescence reader. B. RBD/KRAS stable cells were treated with Tetracycline (1 μg/ml) and AMG510 (0.1 μM) for 24 hours followed by HTRF measurements.

### DETAILED DESCRIPTION OF THE INVENTION

### Definitions

In this specification and in the claims that follow, reference will be made to a number of terms that shall be defined to have the meanings below. All numerical designations, e.g., dimensions and weight, including ranges, are approximations that typically may be varied (+) or (−) by increments of 0.1, 1.0, or 10.0, as appropriate. All numerical designations may be understood as preceded by the term “about”.

Throughout this specification and the claims, the terms “comprise,” “comprises,” and “comprising” are used in a non-exclusive sense, except where the context requires otherwise. Likewise, the terms “include”, “has” and their grammatical variants are intended to be non-limiting, such that recitation of items in a list is not to the exclusion of other like items that can be substituted or added to the listed items. “Consisting essentially of” when used to define systems, compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers. “Consisting of” shall mean excluding more than trace elements of other ingredients and substantial method steps for using the split inteins of this invention. Embodiments defined by each of these transition terms are within the scope of this invention.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range.

The term “about,” particularly in reference to a given quantity, is meant to encompass deviations of plus or minus five percent.

The term “IN” as used herein refers to the N-terminal portion of the intein protein.

The term “IC” is used to refer to the C-terminal portion of an intein protein.

“Bait” as used in this document is a test peptide or polypeptide or protein whose interaction to another peptide, polypeptide or protein (prey as defined below) is being studied.

The term “bait construct” or “bait fusion protein” as used in this document defines a fusion protein between a first test protein or bait peptide (bait), one or more other polypeptides, one of which is IN, and a tag (the tag in the bait fusion protein may be referred to as the “first tag”). The first tag may be located between the first test protein and the IN (bait-tag-IN) or at an end of the IN opposite to the end linked to the bait (i.e. bait-IN-tag).

The term “bait vector” as used in this document refers to a nucleic acid construct which contains sequences encoding the bait construct and regulatory sequences that are necessary for the transcription and translation of the encoded sequences by the host cell, and preferably regulatory sequences that are needed for the propagation of the nucleic acid construct in mammalian cells.

The term “prey construct” or “prey fusion protein” as used in this document defines a fusion between a second test peptide or prey peptide, one or more other polypeptides, one of which is IC, and a tag (the tag in the prey construct may be referred to as the “second tag”). The second tag may be located between the second test peptide and the IN (prey-tag-IC) or at an end of the IC opposite to the end linked to the prey (i.e. prey-IC-tag).

“Prey” as used in this document is a test peptide or polypeptide or protein whose interaction to another peptide, polypeptide or protein (bait, as defined above) is being studied.

The terms “prey vector” and “library vector” as used herein refer to a nucleic acid construct which contains sequences encoding the prey construct and regulatory sequences that are necessary for the transcription and translation of the encoded sequences encoding by the host cell.

The term “tag” as used in this document refers to a nucleic acid sequence or its translation product, which allows the immunological isolation, detection and/or purification of a polypeptide bound to the tag by means of an antibody directed specifically against the tag. Examples of tags that can be used in the present application include V5 tag, HA tag, 3×FLAG tag.

“Test polypeptide” is a polypeptide whose interaction with another polypeptide is being studied with SMPL.

By “isolated” is meant, when referring to a polypeptide, that the indicated molecule is separate and discrete from the whole organism with which the molecule is found in nature or is present in the substantial absence of other biological macro molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

### Overview

The present disclosure relates to a novel approach for protein-protein interaction (PPI) detection that enables a live cell method called Split Intein-Mediated Protein Ligation (SIMPL). In this approach, a split intein is used as a sensor for protein interactions. The present disclosure enables in situ analysis of interactions occurring in various cellular compartments as well as their responses to pharmacological challenges such as enzymatic and PPI inhibitors.

Bait and prey proteins are respectively fused to an intein N-terminal fragment (IN) and C-terminal fragment (IC) derived from a re-engineered split intein GP41-1. The bait/prey binding reconstitutes the intein, which splices the bait and prey peptides into a single intact protein that can be detected by regular protein detection methods such as Western blot analysis and ELISA, serving as readouts of PPIs. The method is robust and can be applied not only in mammalian cell lines but in animal models such as C. elegans. SIMPL demonstrates high sensitivity and specificity, and enables exploration of PPIs in different cellular compartments and tracking of kinetic interactions. Additionally, a SIMPL ELISA platform is disclosed that enables high-throughput screening of PPIs and their inhibitors.

In one embodiment, the re-engineered or artificial split intein of the present disclosure comprises a C-terminus fragment (IC) that includes amino acid residues at positions 13 to 37 of wild type IC of GP41-1, and a N-terminus fragment (IN) that includes amino acid residues at positions 1 to 88 of the wild type IN of GP41-1 fused to amino acid residues 1 to 12 of wild type IC of GP41-1 (C25 GP41-1 split intein), or (ii) the IC includes amino acids at positions 14 to 37 of wild type IC of GP41-1 and amino acid residues at positons 1 to 88 of wild type IN of GP41-1 fused to amino acid residues at positions 1 to 13 of wild type IC of GP41-1 (C24 GP41-1 split intein), or (iii) the IC includes amino acids at positions 15 to 37 of wild type IC of GP41-1 and amino acid residues at positons 1 to 88 of wild type IN of GP41-1 fused to amino acid residues at positions 1 to 14 of wild type IC of GP41-1 (C23 GP41-1 split intein).

### SIMPL

The SIMPL design includes (a) a bait construct or bait fusion protein carrying a bait, and (b) prey construct or a prey fusion protein carrying a prey.

To investigate the interaction of the test proteins in vivo both the bait fusion protein and the prey fusion protein are expressed in a cell line of interest, including mammalian and non-mammalian cells, preferably mammalian cells. If the bait and the prey interact, then the association of the bait in the bait fusion protein and the prey in the prey fusion protein brings IN and IC into close proximity, allowing them to reconstitute into a fully functional intein, which then catalyzes its excision and the concurrent ligation of the bait and the prey (as well as their respective tags) into an intact protein. The resulting spliced protein can be resolved by regular analytical procedures such as Western blot analysis due to its altered mobility, while the presence of the tags allows one or more of visualization, isolation, immobilization or purification of the intact protein using regular biochemical techniques.

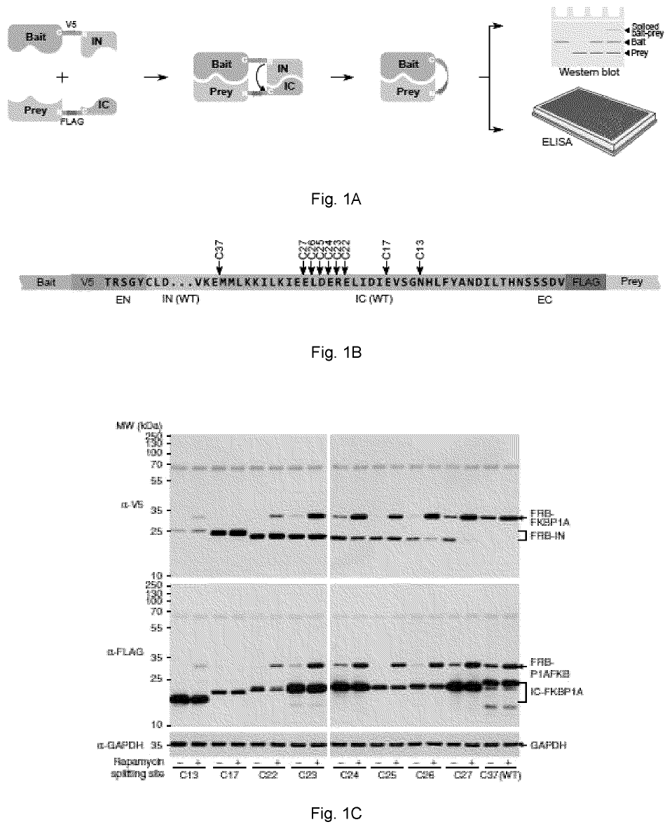

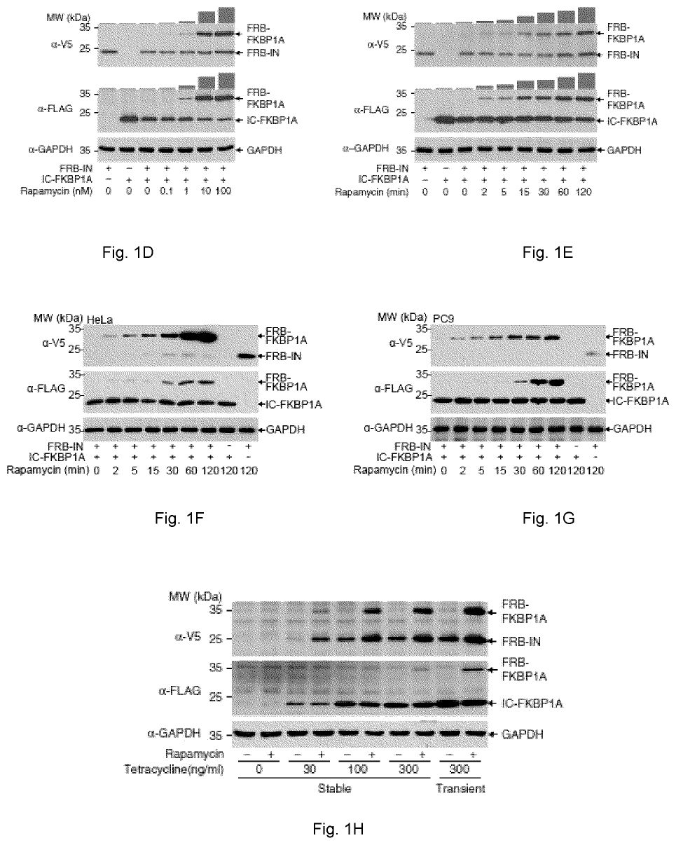

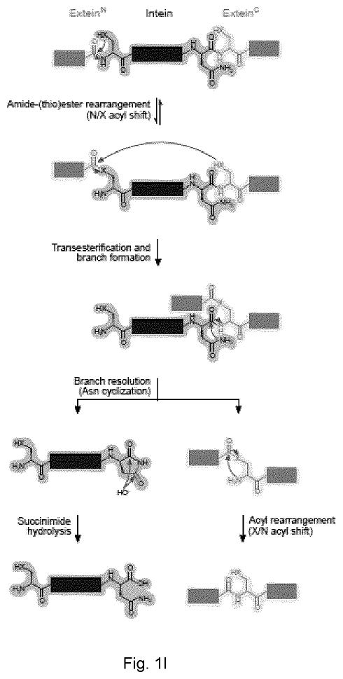

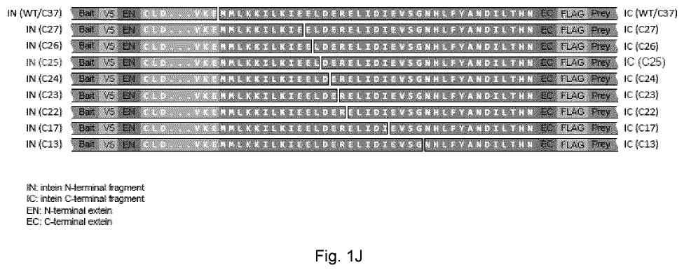

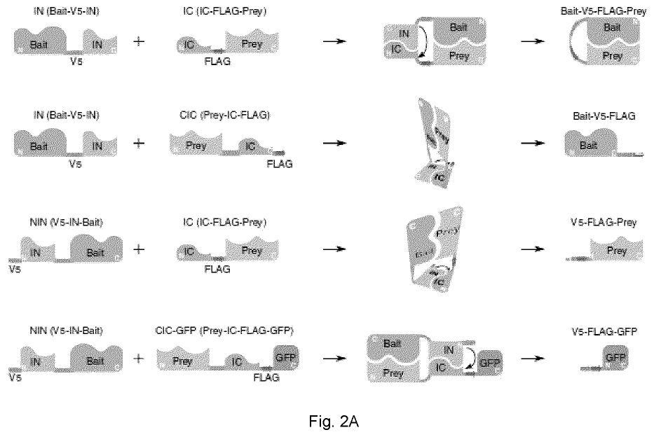

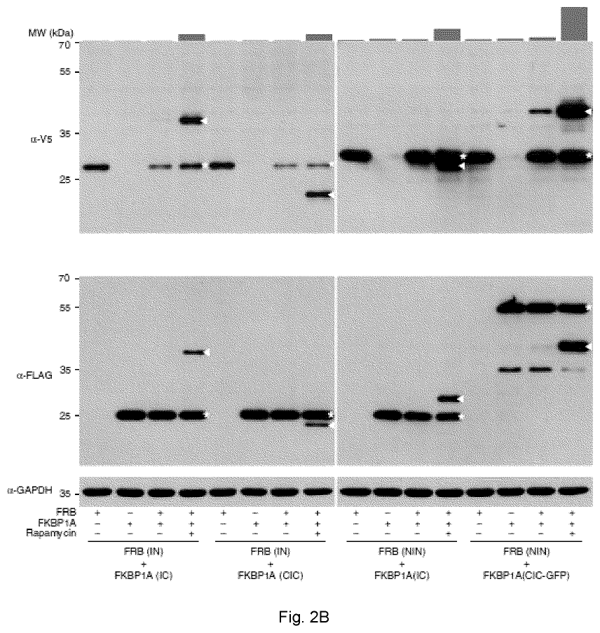

In one embodiment, the bait is connected to the IN at its N-terminus directly, or indirectly through the first tag (see FIG. 1A). In another embodiment, the bait protein is connected to the IN at its C-terminus directly or indirectly through the first tag (see FIG. 2A). The first tag may be fused between the bait and the IN (see FIG. 1A) or the first tag may be fused at a free terminus of the IN (see FIG. 2A).

In one embodiment, the prey protein is connected to the IC at its N-terminus directly, or indirectly through the second tag (see FIG. 1A). In another embodiment, the prey protein is connected to the IC at its C-terminus directly or indirectly through the second tag (see FIG. 2A). The second tag may be fused between the prey and the IN (see FIGS. 1A and 2A) or the second tag may be fused at a free terminus of the IC (see FIG. 2A).

FIG. 1A illustrates an embodiment of a construct in which a bait is fused at its C-terminus to the IN and a prey is fused at its N-terminus to the IC. Two alternative construct arrangements are shown in FIG. 2A. In the C-terminal IC (CIC) prey construct design alternative, the IC moiety is fused to the C-terminus of a prey while keeping the second tag (such as FLAG) downstream. Its interaction with a bait-first tag-IN molecule (in FIG. 2A the first tag is V5) leads to splicing between the bait (as well as the first tag in the bait-V5 tag) and the FLAG tag, which produces a bait-V5-FLAG peptide. In the N-terminal IN bait construct design alternative (NIN) the IN moiety is fused to the N-terminus of a bait while keeping the first tag (such as V5) upstream. When NIN binding to an IC-prey molecule, the N-terminal IN (NIN) bait with an upstream V5 tag produces a V5-FLAG-prey peptide (see FIG. 2A). Since both the CIC and NIN approaches lead to tag transfer, they still provide a readout of interaction that is compatible with Western blot or IP-coupled Western blot analysis. Assessment of these two alternative SIMPL arrangements using the FRB/FKBP1A pair confirmed their feasibility, with rapamycin-induced bands corresponding to the molecular weight of appropriately spliced protein detected via Western blot analysis in all cases (FIG. 2B).

The following is a list of the intact proteins that result from the different combinations of bait construct and prey construct:
IN+IC: intact peptide includes bait-first tag-second tag-prey (see FIG. 1A and top of FIG. 2A)
NIN+IC: intact protein includes first tag-second tag-prey (see FIG. 2A third construct from the top)
IN+CIC: intact protein includes bait-first tag-second tag (see FIG. 2A second construct from the top)

In one embodiment, the disclosure also provides for a CIC-GFP construct (prey-IC-FLAG (second tag)-GFP; see FIG. 2A fourth construct from the top) which can react with NIN bait to produce V5 (first tag)-FLAG-GFP peptide that allows detection by western blot or other suitable analyses.

### HI Throughput Constructs

Use of the SIMPL construct of the present disclosure in combination with an analytical procedure such as Western blot analysis is applicable to detailed PPI analysis, this analysis is limited to low throughput analyses. Western blot analysis, although quantifiable, is not a preferred analytical method to quantify PPI. As such, the present disclosure provides, in another embodiment, for SIMPL constructs that can be used in ELISA for high-throughput, quantifiable measurements of PPI.

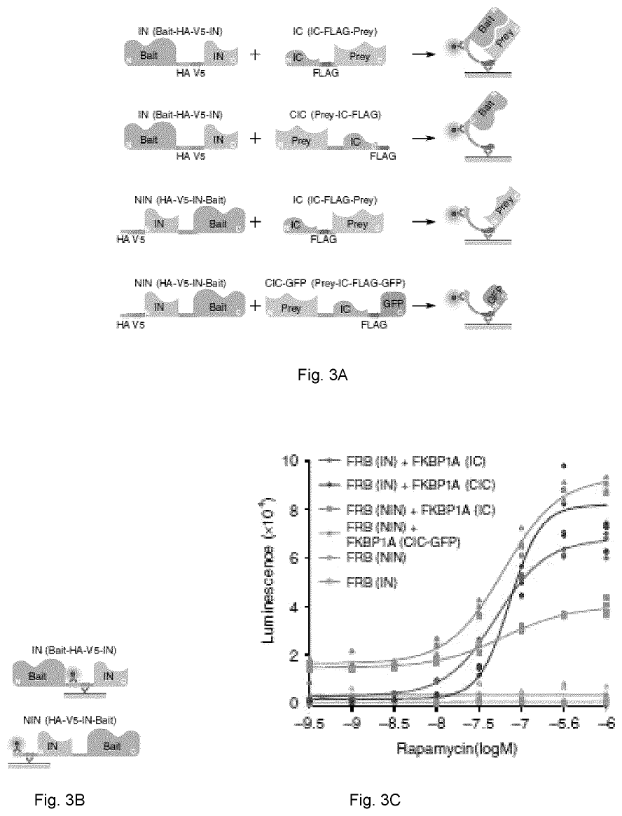

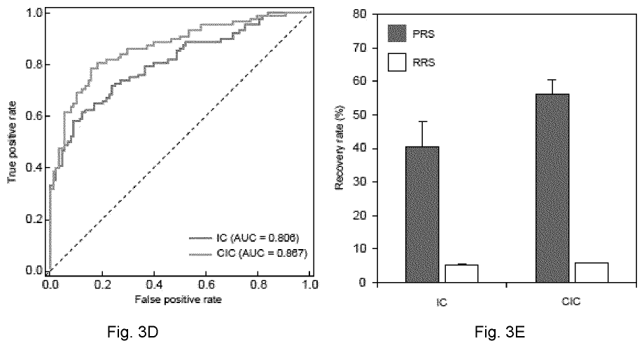

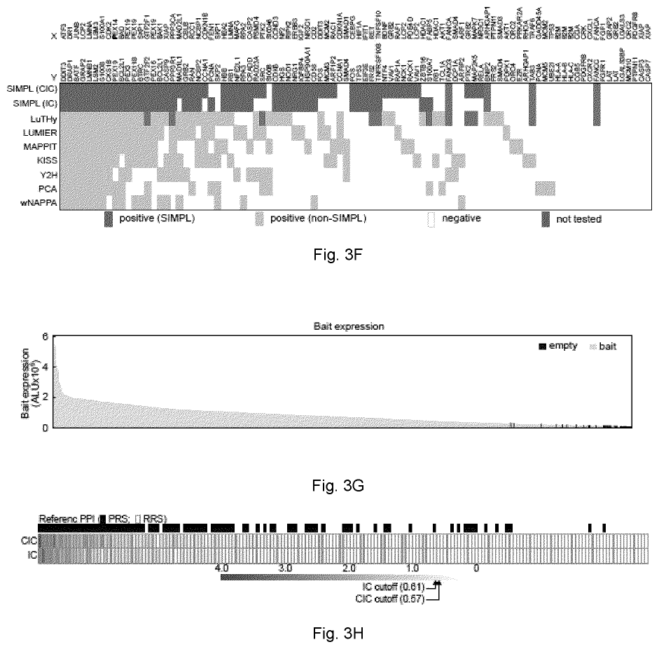

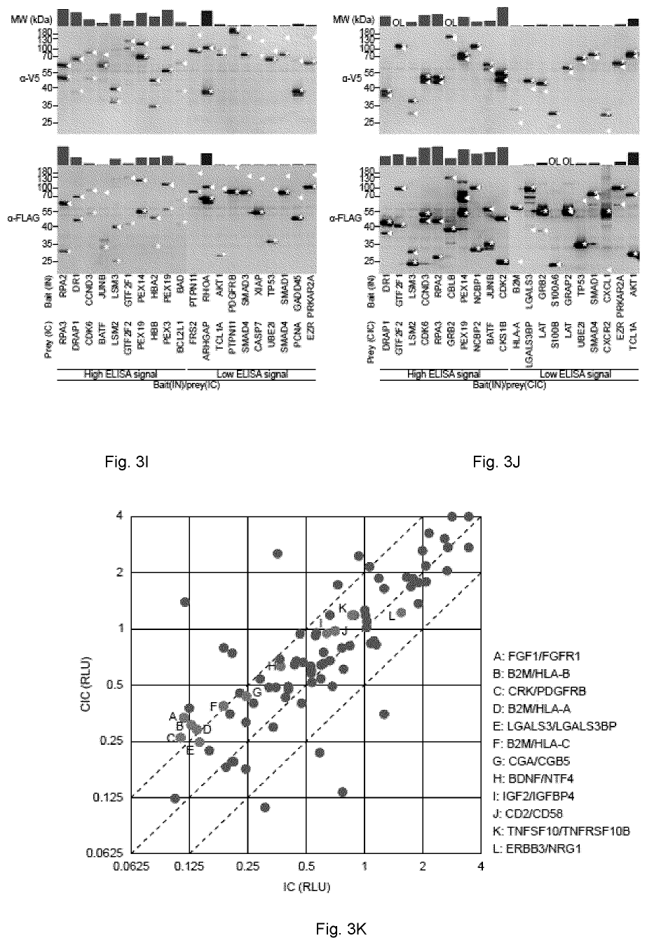

In this embodiment, the bait construct may include two different tags in tandem (see FIG. 3A). In this embodiment another tag (or third tag) such as a hemagglutinin (HA) tag can be inserted into the bait construct in tandem with the first tag. The third tag may be any tag that allows for detection, such as using an anti-third tag antibody coupled to horse radish peroxidase (HRP), while the first tag may allow for immunological immobilization of a protein carrying this first tag to a substrate coated with an anti-first tag antibody. Other possible tags include Strep-tag III and Myc tag.

The constructs and methods of the present disclosure provide for detection of physiological PPIs (see FIG. 3A) and detection and follow up of weak or transient PPIs.

### Applications

The SIMPL constructs of the present disclosure may be used as a high-throughput screening technology for the identification of PPI of any proteins.

In addition SIMPL is sensitive enough to detect subtle changes in protein interactions, which can differ slightly depending on the presence or absence of various stimuli, like hormones or agonists, or inhibitory drugs. Specifically, SIMPL follows the kinetic process of kinase/substrate interactions.

### (2) Drug Screening Platform

SIMPL can be used as a drug screening platform suitable for the identification of small molecule inhibitors or enhancers that alter a defined set of membrane protein interactions in their natural environment.

In another embodiment, the present disclosure provides for a kit of reagents for detecting binding between a first protein (membrane or soluble) or part thereof and a second protein or part thereof (membrane bound or soluble). The kit, in one embodiment, may include: (a) a host cell; (b) a first bait vector (bait), which may be maintained episomally or integrated into the genome of the host cell, comprising a first nucleic acid coding for a bait protein or part thereof, an IN and a first tag, the first bait vector may further include a promoter; (c) a second vector (prey), which may be maintained episomally or integrated into the genome of the host cell, comprising a second nucleic acid coding for a prey protein or part thereof, an IC and a second tag, the second prey vector may further comprise a promoter. In one aspect, the kit further includes (d) a plasmid library encoding second proteins or parts thereof.

### Advantages

The system of the present disclosure presents several advantages over existing techniques to study interactors of membrane proteins: (i) SIMPL can be carried out in virtually any cell line due to the availability of prey/bait/reporter vectors for lentivirus generation, which poses the advantage of single copy integration and diminishes overexpression artifacts. Moreover, SIMPL is carried out in living cells, thus avoiding signal changes arising from cell lysis or protein purification used in biochemical PPI methods; (ii) SIMPL is compatible with ELISA, allowing for a fast, high throughput and quantifiable results; (iii) SIMPL can detect subtle changes in interaction patterns, which can be induced/repressed by either drugs, various stimuli or phosphorylation events, in a highly specific manner; (iv) SIMPL can be used as a platform for drug discovery, specifically used to screen for novel compounds capable of inhibiting signaling mediated by oncogenic receptors; (vi) SIMPL may be used in quantitative studies to measure the strength or affinity of PPI; (vii) as splicing occurs in situ, both loss of specific interaction and gain of nonspecific interaction during processing steps, which are common problems for many affinity-based methods such as co-immunoprecipitation and AP-MS, are avoided; and SIMPL can detect PPIs in various cellular compartments; (viii) SIMPL can be coupled to Homogeneous Time Resolved Fluorescence (HTRF).

In order to aid in the understanding and preparation of the present disclosure, the following illustrative, non-limiting examples are provided.

### Examples

### Materials and Methods

Molecular cloning and library preparation. The plasmids containing GP41-1 split intein cDNA, pCAG-Co-InCreN and pCAG-Co-InCreC, were obtained from Addgene. SIMPL bait and prey vectors are generated by integrating DNA pieces of GP41-1 split intein fragments, linkers, and tags, as well as Gateway cloning cassette, into pCMV5 vector backbone by Gibson assembly (New England BioLabs). Plasmids for FlpIn stable cloning were created similarly into pCDNA5/FRT/TO vector with both bait and prey included by Gibson assembly. Most cDNAs were originally obtained from human ORFeome collection or from the Openfreezer collection at Lunenfeld-Tanenbaum Research Institute [39]. Those not in entry clone vectors were cloned into pDONR223 by PCR and Gateway BP reactions (Life Technologies). Different cDNA fragments were then cloned into SIMPL vectors by Gateway LR reactions (Life Technologies). Site-directed mutagenesis was generated by PCR using KAPA HiFi DNA polymerase (KAPA Biosystems). The plasmids created in this study are available from the corresponding author upon reasonable request.

The natural version (WT) of intein N-terminal fragment (IN) of GP41-1 split intein contains 88 amino acids. Its correspondent (WT) intein C-terminal fragment (IC) contains 37 aa (see FIG. 1B and first top line of FIG. 1I). That is, the WT intein naturally splits at C37. To artificially re-split it, we first fused IN with IC (88+37 amino acids) to create an intact intein, and then split the intact intein at selected sites. In this example, as illustrated in FIGS. 1B and 1n the second to the eighth lines from the top of FIG. 1I) the selected sites were C13, C17, C22, C23, C24, C25, C26, C27, however, other sites may also be selected. The site is named according to the rule in the research field of split intein: numbering from the closest terminus. For example, C25 re-splitting indicates the site between the 26th and the 25th amino acids from the C-terminus. Thus, IC (C25) contains 25 amino acids (aa13-aa37 of WT IC). IN (C25) contains 100 amino acids (aa1-aa88 of WT IN+aa1-aa12 of WT IC).

Cell culture and treatment. HEK 293 and HeLa cell lines were generous gifts from Dr. J. Moffat. HEK 293 Flp-In T-Rex cell line was a generous gift from Dr. A. C. Gingras. These cells were grown in DMEM supplemented with 10% fetal calf serum (Life Technologies). PC9 cell line was a generous gift from Dr. P. Jannes and they were grown in RPMI 1640 medium supplemented with 10% fetal calf serum. For ELISA assay, cells were seeded in 96 well (Sarstedt AG & Co) or 384 well plates (Greiner Bio-One) with 15,000 (96 well) or 5,000 (384 well) cells per well. Cells were transfected with various plasmids with polyethylenimine (PEI) Max (Polysciences) [40]. Expression in HEK 293 Flp-In T-Rex cells was induced by treating the cells with tetracycline (1 μg/ml) for 6-16 hrs. For experiments to study signaling pathway activation, the cells were starved with 0.1% fetal calf serum as well as treated with tetracycline for 6 hrs before stimulation with EGF (Sigma-Aldrich), Tetradecanoylphorbol acetate (TPA) (Sigma-Aldrich) or anisomycin (Sigma-Aldrich). Stable cell lines were created according to the manual of Flp-In T-REx (Invitrogen). Briefly, plasmid containing both bait and prey DNA and pOG44 plasmid (1:10 ratio) were cotransfected into HEK 293 Flp-In T-Rex cells.

After 3 days, the cells underwent puromycin selection. Single colonies were selected and the expression of bait and prey were verified by Western blot analysis.

Western blot analysis and immunoprecipitation. Cells were lysed in buffer H (Triton X 100 1%, β-glycerophosphate pH7.3 50 mM, EGTA 1.5 mM, EDTA 1 mM, orthovanadate 0.1 mM, DTT 1 mM supplemented with protease inhibitors (Roche)). After centrifugation at 21,000×g for 10 min, the supernatants were mixed with Laemmli sample buffer, boiled at 95° C. for 3-5 min and subjected to Western blot analysis. For immunoprecipitations, supernatants (0.3 ml) were incubated with antibodies at 4° C. with rotation for one hour, followed by another hour of incubation with protein G sepharose beads (GE Healthcare). Beads were washed twice with LiCl (0.5 M in Tris pH8.0 0.1 mM) and twice with lysis buffer, boiled with Laemmli sample buffer and then were subjected to Western blot analysis. Antibodies used for Western blot analysis and immunoprecipitations were: α-FLAG antibody purchased from Sigma-Aldrich Co. (F1804) with 1:10,000 dilution and α-V5 antibody from Cell Signaling Technology (#13202) with 1:10,000 dilution. Each of the above antibodies was diluted according to provider's protocol.

ELISA. HEK 293 cells were grown in 96 well or 384 well plates and were transfected with PEI as aforementioned. The cells in each well were lysed in 120 μl (96 well) or 80 μl (384 well) THE buffer (Tris pH7.5 20 mM, NaCl 150 mM, EDTA 2 mM and Triton X-100 0.5% supplemented with protease inhibitors). Aliquots of lysates (20 μl) were incubated for 3 hrs at 40 C in a well of a 384 well Lumitrac plate (Greiner Bio-One) that was coated with α-FLAG antibody (20 μl/well with 1:100 dilution) and blocked with BSA. After 3 times thorough wash with phosphate buffer saline supplemented with 0.05% Tween 20 (PBST), the plate was incubated with HRP-conjugated α-HA antibody (GeneTex GTX115044, 1:5,000 dilution) for 1 hr at room temperature. The plate was washed 3 times with PBST followed by chemiluminescence reading using SuperSignal ELISA Pico substrate (ThermoFisher).

Selection of RRS pairs. All bait-prey pairs (75 baits×78 preys) were considered for the RRS, and 88 were selected that had the lowest chances of interaction, using the following criteria: 1. Absence from the PRS; 2. Absence from the Integrated Interactions Database ver. 2018-11 [41], thereby ensuring that the pairs had not been detected in experimental studies, predicted based on orthology, or predicted by five computational algorithms; 3. Lowest probabilities of interaction according the FpClass PPI prediction algorithm [42]; 4. Maximal coverage of candidate baits and preys.

C. elegans SIMPL vectors and cloning. To facilitate assembly of the expression plasmids, we used a SapI-based cloning strategy. We generated a series of donor vectors based on the kanamycin resistant cloning vector pHSG298 (Takara Bio), in which the insert is flanked with SapI-sites. Digestion with SapI yields overhangs that enable assembly of promoter, ORF, split-intein, and UTR into a destination vector (pMLS257 Addgene #73716) in a single ligation reaction. The following plasmids were generated: i) donor plasmids containing IN (pJRK244), IC (pJRK036), and CIC (pJRK152) split-intein donor sequences. Split-intein amino-acid sequences are identical to the mammalian ELISA compatible split-intein constructs, but are codon optimized for C. elegans and contain an artificial intron. ii) two rps-0 promoter donor plasmids, pJRK001 for assembly with IC and IN, and pJRK151 for assembly with CIC. iii) three unc-54 3′-UTR plasmids: pJRK150 for assembly with IC, pJRK153 for assembly with CIC, and pJRK002 for assembly with IN. ORFs were amplified by PCR from a mixed-stage cDNA library and cloned blunt-ended into vector pHSG298 digested with Eco53kl. Plasmid sequences available upon request. Plasmids used for injection were purified using the PureLink HQ Mini Plasmid DNA Purification Kit (ThermoFisher) using the extra wash step and buffer recommended for endA+ strains.

C. elegans strain and culture conditions. C. elegans strains were cultured under standard conditions (Brenner, S. The genetics of Caenorhabditis elegans. Genetcis 77, 71-94 (1974)). Only hermaphrodites were used and all experiments were performed with animals grown at 20° C. on Nematode Growth Medium (NGM) agar plates seeded with E. coli OP50 bacteria.

Extrachromosomal strain generation. Young adult N2 animals were injected with 20 ng/μl of the prey IC/CIC SIMPL plasmid, 5 ng/μl of the bait IN SIMPL plasmid, 20 ng/μl of a plasmid conferring a dominant RoI phenotype and Hygromycin B resistance (pDD382 Addgene #91830), and 55 ng/μl lambda DNA (ThermoScientific SM0191). Four hermaphrodites were injected for each protein-pair and placed on individual plates. After 2-3 days, Hygromycin B (250 μg/ml) was added to the plates to select for transgenic lines. From each plate a single F2 RoI animal was picked to establish up to four transgenic strains per protein pair, and each was tested for expression of the SIMPL constructs.

C. elegans lysis and ELISA. Mixed-stage animals grown under Hygromycin B (250 μg/ml) selection were washed off with M9 buffer (0.22 M KH2PO4, 0.42 M Na2HPO4, 0.85 M NaCl, 0.001 M MgSO4), and washed two more times with M9 buffer. Samples were then pelleted and resuspended in 100-400 μl of Lysis Buffer (25 mM Tris HCl pH 7.5, 150 mM NaCl, 1 mM EDTA, 0.5% Igepal 630, and 1 tablet/50 ml cOmplete protease inhibitor cocktail (Sigma-Aldrich)). After flash freezing in liquid nitrogen and thawing, samples were sonicated with a Diagenode BioRupter Plus, 5 min high setting: 30 s on/30 s off in a 4° C. water bath. The lysates were then spun at max speed in a tabletop centrifuge at 4° C. for 15 min to clear cellular debris. ELISA was performed as above, but the SuperSignal ELISA pico chemiluminescent substrate was used undiluted (ThermoScientific).

Statistics. Two-tailed Student's t-test with n=3 was used to examine the significance of kinase/substrate and mitochondrial PPIs.

### Results

### Design and Facilitation of SIMPL

In one SIMPL design (FIG. 1A), a bait protein is fused at its C-terminus to a V5 tag and an intein N-terminal fragment (IN). Correspondingly, a prey protein is fused at its N-terminus with a FLAG tag and an intein C-terminal fragment (IC). To investigate the interaction of the bait and prey in vivo both proteins are expressed in a mammalian cell line of interest. The association of bait and prey brings IN and IC into close proximity, allowing them to reconstitute into a fully functional intein, which then catalyzes its excision and the concurrent ligation of the bait and the prey peptides (as well as the V5 and FLAG tags) into an intact protein. The resulting spliced protein can be resolved by regular Western blot analysis due to its altered mobility, while the presence of the V5 and FLAG tags allows visualization or purification of protein using regular biochemical techniques.

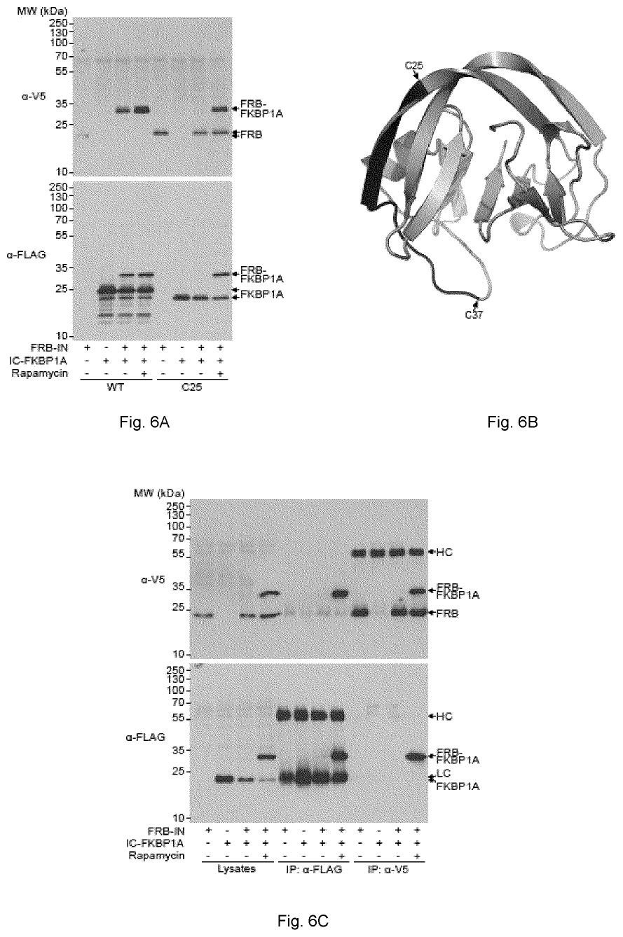

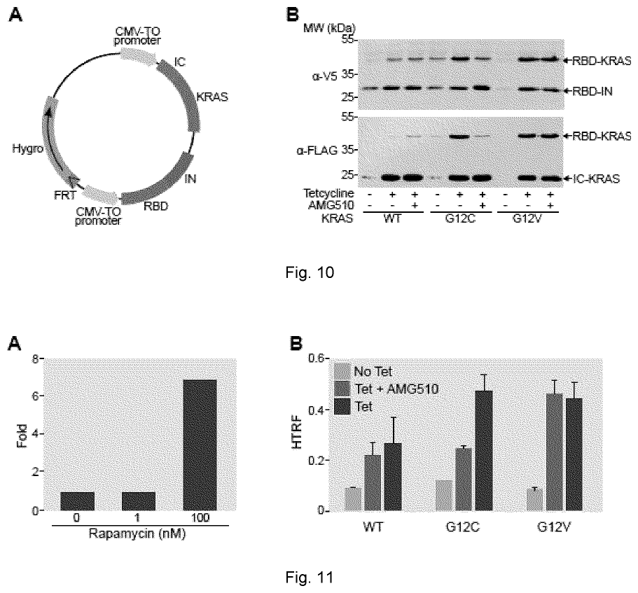

The GP41-1 split intein, which was identified from environmental metagenomics sequence data [9], was chosen for use in the SIMPL system due to its small size (88 amino acids long in IN and 37 amino acids long in IC) and because it possesses the most rapid reaction rate among all split inteins examined [7,10]. Rapamycin-induced heterodimerization of FKBP1A (FKBP12) (IC fused) and FKBP rapamycin-binding (FRB) domain of mTOR11 (IN fused) was used as a test case to evaluate SIMPL performance in a HEK 293 mammalian cell background. Using the WT form of GP41-1, which is split at position C37 (numbered from the last C-terminal amino acid of IC), led to a relatively high level of FRB/FKBP1A splicing even in the absence of rapamycin treatment (FIGS. 10 and 6A), consistent with the known self-association between these IN and IC fragments. To overcome this intrinsic affinity, we re-engineered the GP41-1 split intein. The GP41-1 was re-split at eight different sites (FIG. 1B). Among them, the C25 split intein (FIG. 10 and FIG. 6B) exhibited excellent performance with no apparent loss of enzyme activity and minimal self-association that is hardly detected by Western blot (FIG. 10 and FIG. 6A). The fidelity of the splicing reaction is also demonstrated as only parental and spliced proteins are detected (FIG. 10 and FIG. 6A), suggesting that no N- or C-terminal cleavage occurred, which is a common side reaction of many splitinteins [6, 12]. The identity of the spliced protein was further verified by immunoprecipitation, where the proteins were immunoprecipitated by α-FLAG antibody, stringently washed and probed with α-V5 antibody (or vice versa). In both cases only the spliced protein was detected and no obvious signal was observed in the sample without rapamycin treatment (FIG. 6C). The C25 GP41-1 split intein was therefore adopted for use in our SIMPL system.

To further characterize the SIMPL system, we treated the HEK 293 cells expressing FRB/FKBP1A SIMPL constructs with different concentrations of rapamycin (FIG. 1D). The results showed a typical dose-response relationship with a dose range similar to those measured by BRET-based methods [13]. A time course rapamycin treatment experiment also demonstrated a fast response, with interaction observed in as little as 2 minutes (the smallest observation interval used) and persistently accumulating over time (FIG. 1E). Similar kinetics were also observed in HeLa cells (FIG. 1F) and PC9 lung adenocarcinoma cells (FIG. 1G), suggesting that SIMPL can be applied to different mammalian cell lines. It should be noted that this time series signal profile is distinct from that observed with other methods: time course experiments performed using NanoBRET observed that the interaction rapidly reaches an equilibrium between association and dissociation and maintains a steady state thereafter [13]. This is derived from the differences in what various methods measure. Regular methods such as NanoBRET usually detect PPI complexes themselves. In contrast, as protein splicing is an irreversible process, SIMPL solely measures the event of protein association but does not reflect the dissociation process or the steady state complex.

We further assessed SIMPL in isogenic stable cells to examine potential problems derived from transient transfection such as uneven expression in various cells and difficulty in manipulating expression level. The stable cell line was created by incorporating both FRB-IN and IC-FKBP1A into the genome of host Flp-In T-Rex HEK 293 cells through Flp recombinase-mediated integration. Cells with different expression levels of both FRB and FKBP1A, induced by incubation with varied doses of tetracycline, were treated with rapamycin (FIG. 1H). Interaction-induced splicing and dose-responsive expression of FRB and FKBP1A was observed in all samples, even at the lowest dose of tetracycline (30 ng/ml) employed. Importantly, increasing protein expression was not accompanied by a significant increase in background signal as judged in samples without rapamycin treatment. These observations demonstrate that the SIMPL reaction is efficient, specific and highly sensitive, and can work properly across a wide range of bait and prey expression levels.

Alternative SIMPL formats to extend its detection capability. In the above prototypic SIMPL design, a bait molecule is fused at its C-terminus to the IN and a prey is fused at its N-terminus to the IC. While functional in many cases, this strict arrangement limits the overall detection capability of SIMPL because in some instances the two tags in this format may be spatially inaccessible to each other. Additionally, the function of some proteins may be disrupted by the presence of tags on specific termini, necessitating a different strategy. To address these limitations, we designed alternative intein construct arrangements (FIG. 2A). In the C-terminal IC (CIC or prey-IC-FLAG) format IC is fused to the C-terminus of a prey while keeping the FLAG tag downstream. Its interaction with an IN bait leads to splicing between the bait (as well as V5 tag) and the FLAG tag, which produces a bait-V5-FLAG peptide. Similarly, the NIN (V5-IN-bait) format tags a bait N-terminally with the IN fragment and an upstream V5 peptide. Its interaction with an IC prey produces a V5-FLAG-prey peptide. Since both approaches lead to tag transfer, they still provide a readout of interaction that is compatible with western blot or IP-coupled western blot analysis. However, the interaction between an NIN bait and a CIC prey will produce a spliced small peptide V5-FLAG beyond the detection of western blot analysis. To address this, we created a CIC-GFP construct (prey-IC-FLAGGFP) which can react with NIN bait to produce V5-FLAG-GFP peptide that allows detection by western blot or other analyses. Assessment of all four combinations of different SIMPL arrangements using the FRB/FKBP1A pair confirmed their feasibility, with rapamycin-induced bands corresponding to the molecular weight of appropriately spliced protein detected via western blot analysis in all cases (FIG. 2B, Table 1).

It should be noted that a basal splicing signal appeared in the sample of NIN/CIC-GFP combination without rapamycin treatment. This might be derived from an affinity change caused by different tagging or high-level expression of the proteins. However, the corresponding rapamycin-treated sample shows a dramatically increased signal (more than eight fold by density), making the states easily distinguishable from one other when proper controls and quantification are used. Thus, all four combinations are suitable for use in PPI detection in SIMPL.

ELISA platform of SIMPL assay and its unbiased evaluation. While use of SIMPL with a Western blot readout is applicable to detailed PPI analysis, it is limited to low-throughput analyses and is not strongly quantifiable. We therefore developed an alternative, ELISA-coupled SIMPL platform for high-throughput, quantifiable measurement of PPIs. For this purpose, a hemagglutinin (HA) tag was introduced into the bait construct in tandem with V5. This allows for monitoring of protein splicing using an ELISA format, with protein capture performed using α-FLAG antibody and detection performed using α-HA antibody coupled to horseradish peroxidase (HRP) (FIG. 3A). The SIMPL signal can be normalized to bait expression, which can be similarly measured by ELISA using immobilization with α-V5 antibody followed by detection with HRP conjugated α-HA antibody (FIG. 3B). Performance of the ELISA platform was tested by monitoring the dose-response of rapamycin-induced FRB/FKBP1A interaction in all four formats: FRB-IN/IC-FKBP, FRB-IN/FKBP-CIC, NIN-FRB/IC-FKBP and NIN/CIC-GFP (FIG. 3C). The results of all four formats showed an expected dose-response relationship similar to that obtained by Western blot analysis (FIG. 2B), demonstrating the feasibility of SIMPL ELISA. Specifically, the IN/IC profiles obtained from both ELISA analysis and quantified Western analysis (FIG. 1D) presented notable similarity. Although two combinations, NIN/IC and NIN/CIC-GFP, showed relatively elevated basal splicing levels, these background signals were not high enough to interfere with interpretation of the spliced signal induced by rapamycin, with a robust 1.5 fold and 4 fold increase observed in the NIN/IC and NIN/CIC-GFP formats in ELISA analysis, respectively, after rapamycin treatment. In addition, expression of bait alone, either FRB (IN) or FRB (NIN), did not show any response to rapamycin treatment, further demonstrating the specificity of the assay. The application of SIMPL coupled to an ELISA-based detection platform allows high-throughput PPI screening with a potentially expanded dynamic range of detection.

We next evaluated the SIMPL system using a benchmarking approach with unbiased PPI reference sets, which has been widely accepted for assessing the overall performance of a PPI method. We employed a positive reference set (PRS) which contained 88 available positive PPIs derived from the previously well-established human PRS (hPRS)15, including different types of PPIs and covering those occurring in various subcellular locations (Table 2). Our random reference set (RRS) contained 88 protein pairs with baits and preys selected from the PRS but used in combinations determined computationally to have low probability of interaction (Table 2). The reference sets were evaluated with the ELISA platform in two formats, bait(IN)/prey(IC) and bait(IN)/prey(CIC) (Table 3). The expression of baits in the same samples was also tested with ELISA (FIG. 3G) and each SIMPL signal was thereby normalized to its corresponding bait expression (FIG. 3H). Receiver operating characteristic (ROC) analyses of the assays demonstrated exceptional sensitivity with AUC values of 0.806 and 0.867 for IN/IC and IN/CIC formats, respectively (FIG. 3D). Accordingly, 41% (IN/IC) or 56% (IN/CIC) of PPIs can be detected by SIMPL without compromising assay specificity and maintaining a false-positive rate of ˜5% (FIG. 3E), determined with threshold values obtained from ROC analyses. We further compared the ELISA readout with Western blot analysis by selecting 10 PPIs with high ELISA signals (above threshold) and 10 with low ELISA signals (below threshold) either in IN/IC or IN/CIC format and re-testing them by Western (FIGS. 31 and 3J). The results were highly consistent between the two methods; nine PPIs in the high IC group and all PPIs in high CIC group presented clearly observable splicing bands. In contrast, only one PPI in each low signal group presented a strong splicing signal relative to the levels observed in the high group bands. Comparison with the results from other PPI methods in the literature [15-17] shows marked improvement of detection (FIG. 3F). Interestingly, although many PPIs can be detected by SIMPL using both the IN/IC and IN/CIC formats, they do show differences between them, likely due to the spatial geometry of the interacting molecules or due to the disruption of the functionalities of the tagged termini. In general, the CIC format exhibits better performance in tests described here (FIG. 3D and FIG. 3K). For example, prey proteins containing signal peptides showed better detectability in CIC format (FIGS. 3D and 3K) since tagging on N termini blocks their recognition by signal recognition particle and thereby affects their correct sorting to the membrane.

Further Characterization of SIMPL with PPIs Involved in Signaling Pathways and Mitochondrial Biogenesis.

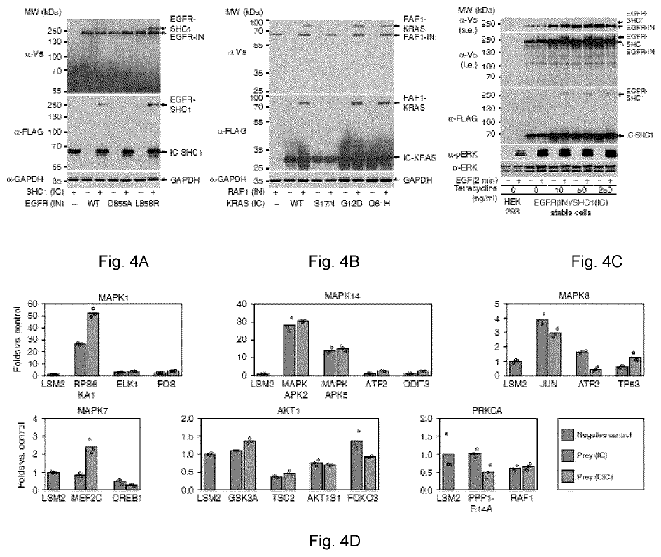

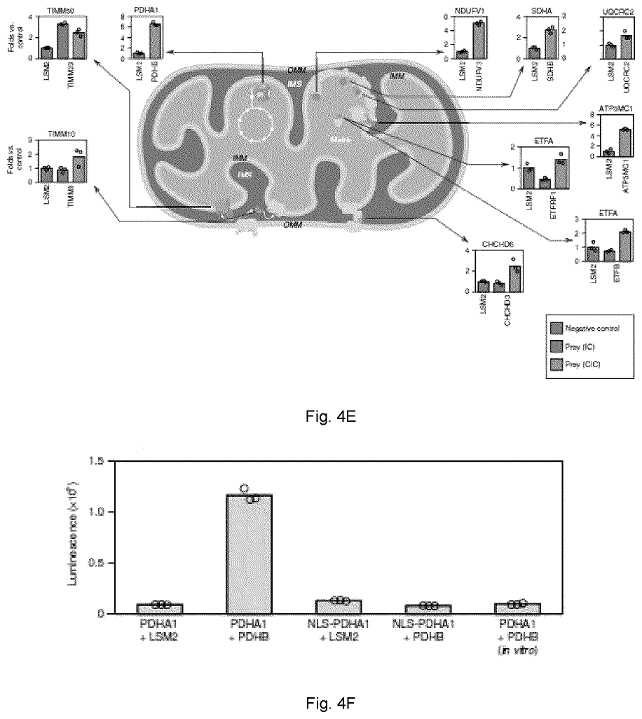

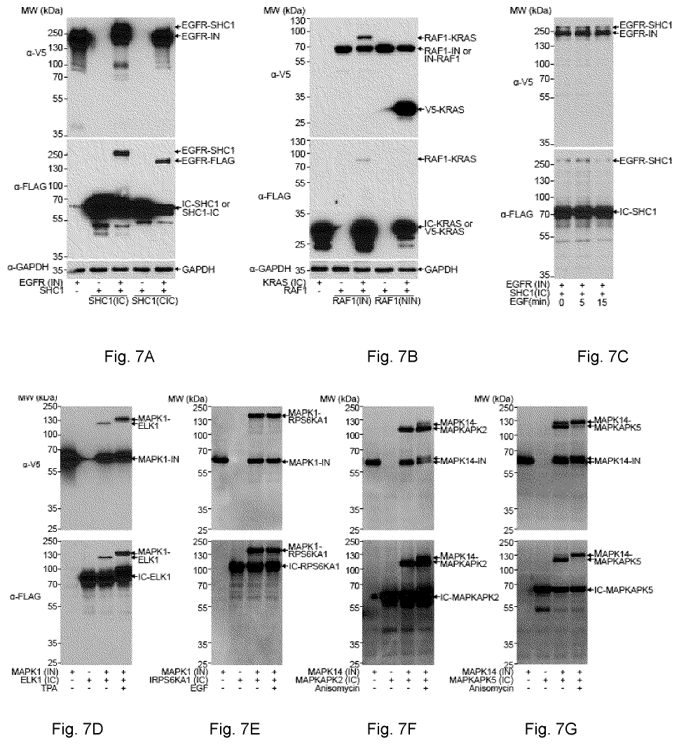

We next used SIMPL to explore physiological PPIs and chose PPIs in the EGFR-RAS-ERK1/2 axis, an important signaling pathway involved in multiple physiological and pathological processes [18]. Activated EGFR undergoes autophosphorylation on tyrosine residues, which recruit scaffold proteins such as SHC1 to relay signal to downstream machinery [19]. At the RAS level, activated RAS (KRAS in this study) binds and directly activates RAF kinases20 (RAF1 in this study). When EGFR-IN and IC-SHC1 were co-expressed in HEK 293 cells, their interaction was captured as a spliced band above EGFR recognizable by both α-FLAG and α-V5 antibodies (FIG. 4A). The EGFR/SHC1 PPI was also effectively detected using SHC1-CIC construct via transfer of FLAG to EGFR bait (FIG. 7A). The interaction depends on EGFR activity as the constitutively active EGFR mutant (L858R) enhanced the signal while the kinase dead mutant (D855A) [21, 22] abolished the interaction (FIG. 4A). To detect KRAS/RAF1 interaction, we chose the IC-KRAS construct to avoid its C-terminal tagging as KRAS protein undergoes C-terminal lipidation for its membrane anchoring. Assay with RAF1-IN detected the specific interaction (FIG. 4B) as wild type KRAS and its constitutively active mutants (G12D and Q61H) displayed splicing signals, while no obvious signal was observed with the dominant negative KRAS mutant (S17N)23. Assay with the NIN-RAF1 construct exhibited a more marked splicing signal than RAF1-IN (more than 6 fold by density), which was derived from V5 tag transfer to the interacting KRAS protein (FIG. 7B). We speculate that the signal enhancement is caused by RAF1 N-terminal tagging, which improved accessibility of reactive termini since the RAS-binding domain is located at the N-terminus of RAF1. Thus, these two classical PPIs involved in normal and oncogenic signaling were successfully recapitulated by SIMPL.

As the study of rapamycin-induced FRB/FKBP1A interaction demonstrated the potential of SIMPL to follow interaction kinetics (FIG. 1E and FIGS. 1F-1G), we tested whether SIMPL can also track kinetics of physiological PPIs using the example of EGF-activated SHC1 recruitment to EGFR. We found that interaction (splicing) between EGFR (IN) and SHC1 (IC) had already occurred without EGF treatment when they were transiently overexpressed in cells (FIG. 4A), and the signal was only slightly enhanced after EGF stimulation (FIG. 7C) as the density ratio of the spliced bands between non-treated and EGF-stimulated (5 min) samples is 1:1.7. It should be noted that overexpression of EGFR induces ligand independent autoactivation and subsequent recruitment of SHC1 (Jura, N. et al. Mechanism for Activation of the EGF Receptor Catalytic Domain by the Juxtamembrane Segment. Cell 137, 1293-1307 (2009)). Unlike phosphorylation and PPI, splicing is irreversible and therefore led to accumulation of the spliced protein. This accumulated spliced protein likely masked the signal induced by EGF stimulation. The key to overcoming this is to reduce the level and duration of bait and prey expression. Thus, we created a stable cell line derived from Flp-In T-Rex HEK 293 cells by incorporating both EGFR (IN) and SHC1 (IC) into the genome through Flp recombinase-mediated integration. This allowed control of the amplitude and temporal induction of their expression by tetracycline. Notably, in cells treated for 8 hours with tetracycline in starvation medium (0.1% fetal calf serum) the splicing signal of EGFR/SHC1 interaction was markedly reduced, but restored upon EGF stimulation for 2 minutes (FIG. 4C), supporting the feasibility of using SIMPL to follow physiological PPIs. It should be noted that, due to the leakage of the tetracycline repressor system, low levels of EGFR (IN) and SHC1 (IC) were observed in cells without tetracycline treatment. EGF-stimulated splicing was also observed under this condition, consistent with the strong sensitivity of SIMPL assay.

As most PPI methods are not well suited for detection of weak or transient PPIs, we wanted to test whether SIMPL is capable of following these types of interactions. We selected protein kinases as our test case since their association with substrates, similar to common enzyme/substrate interactions, is usually characterized as transient and weak [24]. Thus, IN-fused MAPK1 (ERK2), MAPK8 (JNK1), MAPK14 (p38α MAPK), MAPK7 (ERK5), AKT1 and PRKCA (PKCα) were examined with several well documented substrates in both IC and CIC formats. ELISA assay demonstrated that more than 50% of these PPIs could be detected by SIMPL, in one or both formats (FIG. 4D). MAPK members presented relatively strong interactions, which may be enhanced by docking interaction outside of their active sites [25]. As SIMPL displays an excellent ability to follow the kinetics of the rapamycin-induced FRB/FKBP interaction (FIG. 1E), we examined whether it could also capture the kinetic process of kinase/substrate interactions. Indeed, we observed an enhanced MAPK1/ELK1 interaction after MAPK1 activation induced by tetradecanoylphorbol acetate (TPA) stimulation (FIG. 4D). However, no obvious increase was observed in many cases of kinase activation (FIG. 7E-G). In these instances the accumulation of the spliced proteins from the basal state may have masked the stimulation response due to the irreversibility of the splicing reaction and might be avoided by reducing basal bait/prey expression through the use of the stable cell line approach mentioned above for EGFR/SHC1, an area which deserves further investigation.

The evaluation with the reference PPI set suggests SIMPL is capable of detecting PPIs in various cellular compartments as the PRS covers PPIs occurring in different locations such as nucleus, cytoplasm, plasma membrane and extracellular space (Table 2). We further tested this by examining mitochondrial PPIs with SIMPL as mitochondria are special organelles with distinct features and their PPIs are often difficult to study. We selected several well-studied mitochondrial PPIs for this purpose (Table 4), including proteins involved in oxidative phosphorylation [26], transport [27], cristea organization [28] and metabolism [29]. Baits were prepared using the IN format to avoid interference of transit peptides usually at N-termini of mitochondrial proteins. The corresponding preys were constructed in either the IC or CIC configuration (or both), to reduce the chance of steric interference preventing their association with IN or to prevent incorrect sorting of the prey proteins. Out of 10 PPIs examined, eight (TIMM50/TIMM23, PDHA1/PDHB, CHCHD6/CHCHD3, NDUFV1/NDUFV3, SDHA/SDHB, UQCRC2/UQCRC2, ATP5MC1/ATP5MC1 and ETFA/ETFB) were successfully detected (FIG. 4E), including proteins localized to different sub-mitochondrial compartments (matrix, inner membrane and intermembrane space).

We tested two additional controls for PDHA1/PDHB interaction to exclude the possibility that splicing occurred in cell lysates during sample processing (FIG. 4F). In the first control, the mitochondrial targeting sequence of PDHA1 was replaced with the nuclear localization signal of MYC. This modification prevents the resultant NLS-PDHA1 from being sorted to the mitochondria and, consistent with this, its interaction with PDHB was therefore not observed. In the second control, PDHA1 and PDHB were separately expressed in different cells and the two cell lysates were mixed and assayed by ELISA. Once again, no interaction between PDHA1 and PDHB was detected. Together, these results indicate that a significant amount of PDHA1/PDHB SIMPL splicing does not occur if both proteins are not properly targeted to the mitochondrion, supporting the broad applicability of SIMPL for monitoring interactions with diverse subcellular localizations.

These results display the broad applicability of SIMPL for monitoring interactions with diverse subcellular localizations.

Detecting PPIs in C. elegans with SIMPL

We next investigated the feasibility of using the SIMPL system in a multicellular animal using the nematode Caenorhabditis elegans as a model. We selected a C. elegans PRS of 27 PPI pairs from previously identified literature confirmed interactions [43] and from interactions used previously to evaluate binary PPI mapping approaches [44] (Table 5). We also assembled a C. elegans RRS by randomly combining protein pairs from the PRS, excluding known interactors. Full-length ORFs of the corresponding genes were PCR amplified and cloned into vectors containing split-intein tags optimized for expression in C. elegans, but otherwise identical to the ELISA compatible split-intein fragments used above.

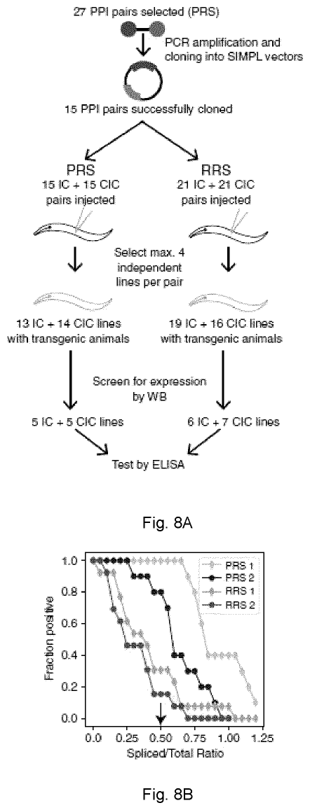

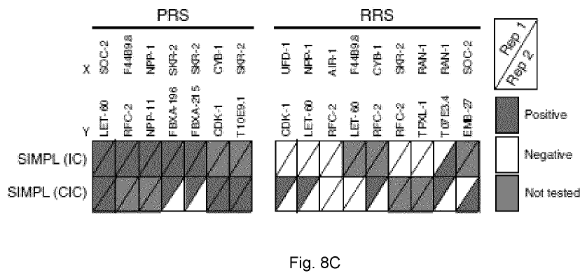

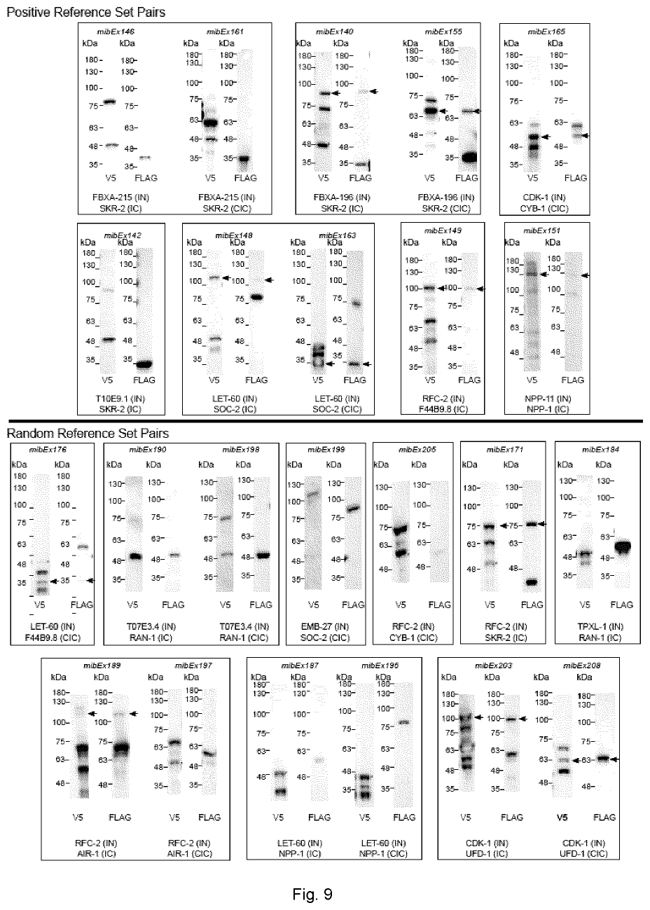

Proteins were expressed under control of the general rps-0 ribosomal promoter. Transgenic animals were generated by microinjection, and both IC and CIC configurations were injected for each prey protein. To enable accurate quantification of splicing by ELISA, we injected a 4× higher concentration of prey plasmid than bait plasmid to ensure that splicing of the bait protein is not limited by the availability of the prey protein. All transgenic lines were first tested for expression of both bait and prey protein by Western blotting. In all, we recovered 10 PRS pair-expressing lines and 13 RRS pair-expressing lines, representing 7 and 9 unique protein pairs respectively (FIG. 8A). We first analyzed each line for splicing by Western blot (FIG. 9). We observed visible splicing for 7/10 PRS pairs and 5/13 RRS pairs, however levels of splicing for the RRS pairs were lower than for the PRS pairs. We then quantified levels of splicing using ELISA as above, analyzing two independent protein lysates for each transgenic line. Using a sliding cutoff level of spliced bait/total bait signal to assign positive interactions, we observed clear separation between PRS and RRS protein pairs in both replicates (FIG. 8B). At a cutoff level that optimizes the fraction of true positives vs. false positives detected in both replicates, 8/10 PRS pairs tested positive in both replicates, while the remaining 2 pairs tested positive in one replicate (FIG. 8C). In contrast, only one of the RRS pairs tested positive in both replicates, and 4 pairs tested positive in a single replicate. Collapsing IC/CIC orientations, 7/7 PRS protein pairs tested positive, and 1/9 RRS pairs tested positive. Overall, these results indicate that the SIMPL system is functional in C. elegans and suggests its universal nature could allow it to be exploited in many other systems.

### SIMPL as a Drug Screening Platform.

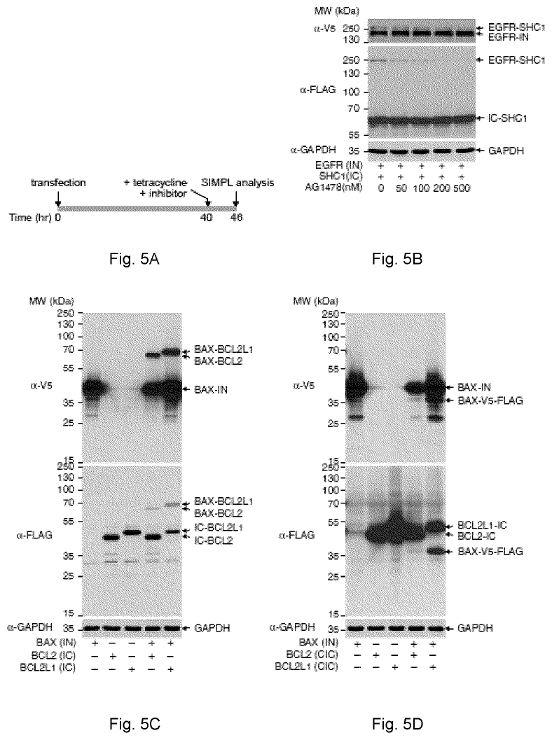

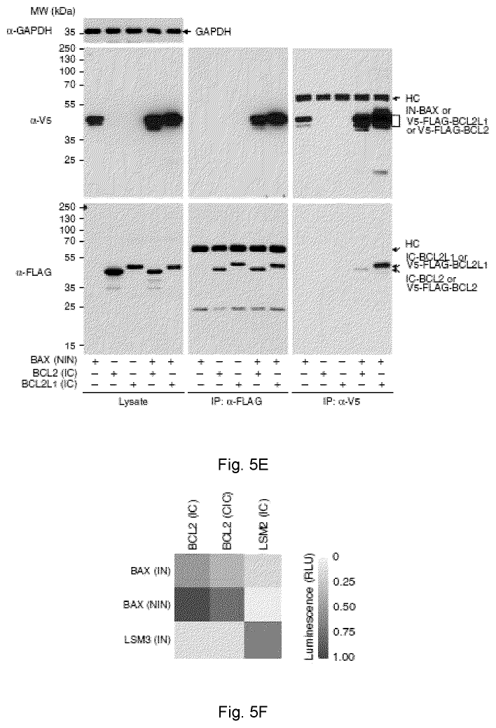

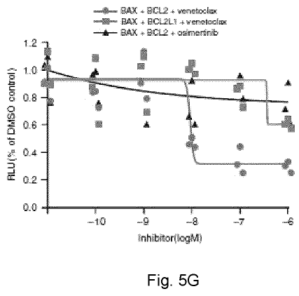

We investigated whether SIMPL can serve as a drug screening tool, in particular as an assay that could detect enzymatic- as well as PPI-inhibitors. Since protein splicing is an irreversible process, inhibitors have to be administered before bait/prey expression (FIG. 5A). Using the EGFR/Shc1 interaction as an example, we observed a decrease of SIMPL signal upon the administration of AG147830, an EGFR tyrosine kinase inhibitor (TKI) which suppresses EGFR autophosphorylation and thereby reduces EGFR/Shc1 interaction (FIG. 5B). In this case, the interaction between Shc1 and EGFR occurs downstream to EGFR activation and serves as an indirect readout of EGFR activity. We then tested whether SIMPL can also monitor PPI inhibition caused by a direct PPI inhibitor using venetoclax as an example, an FDA approved drug targeting the BAX/BCL2 interaction [31], as an example. We first examined the interactions of BAX/BCL2 (and BAX/BCL2L1 control) in all three SIMPL formats, IN/IC, IN/CIC and NIN/IC (FIG. 5C-5F). Note that for the NIN-BAX/IC-BCL2 combination, immunoprecipitation was performed to resolve spliced protein product from the similarly-sized parental product (FIG. 5E). Based on these results the NIN-IC pairing displayed the highest signal (FIG. 5F). Using this combination in SIMPL with an ELISA readout we observed potent inhibition of the BAX/BCL2 interaction in the presence of venetoclax with an IC50 of 9.1 nM (very similar to other cell-based assay results [32]) but not the unrelated control TKI osimertinib (FIG. 5G, (curve with circles) and (curve with triangles)). The effect of venetoclax on BCL2L1 (BCL-XL) was much less potent as the inhibition of the BAX/BCL2L1 PPI was observed only at high venetoclax concentration (FIG. 5G, green (curve with squares)), consistent with previous reports [32]. Overall, these results clearly demonstrate the potential for using SIMPL as a highly sensitive small molecule screening tool.

### Development of Screen System for Protein-Protein Interaction (PPI) Inhibitor

Previously we have shown that SIMPL can serve as an assay to verify activity of different PPI inhibitors. However, this demonstration was carried out by expressing the targeted proteins transiently in the cells, and testing activity of EGFR inhibitor AG1478 and BCL2 inhibitor Veneto lax. The transient ectopic expression restricts the system from being applicable to large scale screening. To overcome this limitation, we created stable cell lines by integrating target genes into the genome of the host cells. This is showcased using KRAS. More specifically, the expression plasmid with open reading frames of both KRAS and Ras binding domain (RBD, derived from Raf1 and responsible for binding to Ras) under the control of T-Rex promoter was created (FIG. 10A). The DNA was inserted into the FRT site of host HEK 293 Flp-In T-Rex cells with the standard Flp-In procedure. Expression of KRAS and RBD was induced by Tetracycline and verified using Western blot analysis (FIG. 10B). Three cell lines with different types of KRAS: WT, G12C and G12V mutants, were created. The interaction between KRAS and RBD was nicely shown by the appearance of spliced bands. The densities of the bands are strongly correlated with the activities of different types of KRAS; oncogenic G12C and G12V mutants showing stronger signals. Furthermore, we evaluated the system using AMG510, a G12C KRAS specific inhibitor, recently developed by Amgen. The results did recapitulate the efficacy and specificity of AMG510: only the interaction of G12C KRAS with RBD was inhibited, while WT and G12V mutant were spared (FIG. 10B). These cell lines will be used in the future to screen inhibitors targeting G12V mutant.

### SIMPL Coupled to HTRF Assay

ELISA is currently used as the readout for medium to high throughput SIMPL assay. However, the tedious procedures that accompany use of ELISA make it less satisfactory. We decided to improve the performance of our assays by coupling SIMPL with Homogeneous Time Resolved Fluorescence (HTRF). In this assay, cell lysates are incubated with antibodies conjugated with fluorescent dyes (Tb as fluorescence donor and d2 as acceptor). After incubation, the splicing signal is read as delayed Fluorescence Resonance Energy Transfer (FRET). Since the emission of the donor dye is relatively sustainable, the real FRET signal can be obtained by delayed measurement and the background signals are filtered out due to their fast emission. The feasibility of HTRF readout platform was demonstrated using Rapamycin-induced FRB/FKBP1A interaction (FIG. 11A). More specifically, the HEK 293 cells were transfected with FRB-IN and IC-FKBP1A and subsequently treated with Rapamycin (0, 1 or 100 nM) for 2 hours. The cells were lysed and incubated with α-FLAG-Tb and α-HA-d2 antibodies at 40 C for 4 hours followed by reading using a fluorescence plate reader compatible with HTRF. The results (FIG. 11A) clearly demonstrate a good separation of the interaction signal (the sample treated with 100 nM Rapamycin) from background signal (the sample without Rapamycin treatment).

Next, we evaluated the HTRF-coupled system using AMG510 to inhibit KRAS(G120)/RBD interaction. The cells with stable integration of KRAS (WT and mutants) and RBD genes (as described in the previous section) were treated with both Tetracycline and AMG510 overnight followed by HTRF assay. The inhibition of KRAS/RBD interaction by AMG510 was only observed in G12C mutant, and not in WT nor G12V mutant. We therefore conclude that HTRF can be used as a powerful readout platform for SIMPL.

TABLE 2
Reference sets for evaluating SIMPL assay
(a) Positive reference set (PRS)

| bait | | prey | |
|---|---|---|---|
| protein | subcellular | protein | subcellular |
| name | localization | name | localization |
| AKT1 | cell membrane | PDPK1 | plasma membrane |
| | (associated), | | (peripheral), |
| | cytoplasm, nucleus | | nucleus, cytosol |
| AKT1 | cell membrane | TCL1A | nucleus, ER |
| | (associated), | ARFIP2 | plasma membrane, |
| | cytoplasm, nucleus | | cytosol, |
| ARF1 | Golgi apparatus | | Golgi apparatus |
| | (anchored), | DDIT3 | nucleus |
| | plasma membrane | HLA-A | plasma membrane, |
| | (anchored) | | ER, Golgi apparatus |
| ATF3 | nucleus | HLA-B | plasma membrane, |
| B2M | extracellular | | ER, Golgi apparatus |
| B2M | extracellular | HLA-C | plasma membrane, |
| B2M | extracellular | | ER, Golgi apparatus |
| BAD | mitochondrion (outer | BCL2L1 | mitochondrion, |
| | membrane), cytoplasm | | cytoskeleton, |
| BAK1 | mitochondrion (outer | | nucleus, cytosol |
| | membrane) | BCL2L1 | mitochondrion, |
| BDNF | extracellular | | cytoskeleton, |
| CASP2 | cytosol, | | nucleus, cytosol |
| | mitochondrion, | NTF4 | extracellular |
| | nucleus | CRADD | nucleus, cytosol |
| CBLB | cytoplasm | GRB2 | plasma membrane, |
| CCND3 | nucleus | | cytoplasm, nucleus |
| CD2 | cell membrane | CDK6 | nucleus, cytoskeleton, |
| CDK2 | nucleus, cytoplasm, | | cytosol |
| | cytoskeleton, | CD58 | plasma membrane |
| | endosome | CKS1B | nucleus |
| CDKN1A | nucleus | CCNA1 | nucleus |
| CDKN1B | nucleus, endosome | CCNA1 | nucleus |
| CEBPG | nucleus | FOS | nucleus, ER, cytosol |
| CGA | extracellular | CGB5 | exracellular |
| CRK | cytoplasm, nucleus, | PDGFRB | plasma membrane, |
| | membrane (associated) | | lysosome |
| CXCL1 | extracellular | CXCR2 | plasma membrane |
| DDIT3 | nucleus | FOS | nucleus, ER, cytosol |
| DR1 | nucleus | DRAP1 | nucleus |
| ERBB3 | plasma membrane | NRG1 | membrane, |
| FABP5 | nucleus, cytosol, | | extracellular |
| | membrane, | S100A7 | extracellular, |
| | extracellular | | cytoplasm, nucleus |
| FEN1 | nucleus | PCNA | nucleus |
| FGF1 | extracellular, cytosol, | FGFR1 | plasma membrane |
| | nucleus | PCNA | nucleus |
| GADD45A | nucleus | LAT | plasma membrane |
| GRAP2 | endosome, cytosol, | LAT | plasma membrane |
| | nucleus | PTK2 | plasma membrane |
| GRB2 | plasma membrane, | | (associated), |
| | cytoplasm, nucleus | | cytoskeleton, nucleus, |
| GRB2 | plasma membrane, | | cytosol |
| | cytoplasm, nucleus | VAV1 | cytosol, plasma |
| GRB2 | plasma membrane, | | membrane |
| | cytoplasm, nucleus | GTF2F2 | nucleus |
| GTF2F1 | nucleus | HBB | cytosol |
| HBA2 | cytosol | RB1 | nucleus |
| HDAC1 | nucleus | ZBTB16 | nucleus |
| HDAC1 | nucleus | TP53 | nucleus, |
| HIF1A | nucleus, cytosol | | mitochondrion, |
| IFIT1 | cytoplasm | | ER |
| IGF2 | extracellular | EIF3E | nucleus, cytosol |
| JUNB | nucleus | IGFBP4 | extracellular |
| LCP2 | cytoplasm | BATF | nucleus |
| LCP2 | cytoplasm | GRAP2 | endosome, cytosol, |
| LCP2 | cytoplasm | | nucleus |
| LGALS3 | extracellular, plasma | NCK1 | plasma membrane, |
| | membrane, nucleus, | | ER, nucleus, cytosol |
| | cytoplasm | VAV1 | cytosol, plasma |
| LMNA | nucleus | | membrane |
| LMNA | nucleus | LGALS3BP | extracellular, nucleus, |
| LSM3 | nucleus | | plasma membrane |
| MAD2L1 | nucleus, cytoskeleton, | LMNB1 | nucleus |
| | kinetochore | RB1 | nucleus |
| MAFG | nucleus | LSM2 | nucleus |
| MAPK7 | nucleus, cytoplasm | MAD1L1 | cytoskeleton, nucleus, |
| MCM2 | nucleus | | kinetochore |
| MCM2 | nucleus | NFE2L1 | ER, nucleus |
| NCBP1 | nucleus | MAP2K5 | nucleus, cytoplasm |
| NF2 | plasma membrane | MCM3 | nucleus |
| | (associated), nucleus | MCM5 | nucleus, cytosol |
| NR3C1 | mitochondrion, | NCBP2 | nucleus |
| | cytosol, nucleus, | HGS | endosome, cytoplasm |
| | cytoskeleton | HSP90AA1 | plasma membrane, |
| NR3C1 | mitochondrion, | | cytoplasm, nucleus |
| | cytosol, nucleus, | RELA | nucleus, cytoplasm |
| | cytoskeleton | MCM10 | nucleus |
| ORC2 | nucleus | ORC4 | nucleus |
| ORC2 | nucleus | RACK1 | plasma membrane, |
| PDE4D | plasma membrane | | nucleus, |
| | (associated), | | mitochondrion, |
| | cytoskeleton | | cytosol |
| PDGFRB | plasma membrane, | PTPN11 | nucleus, cytoplasm |
| | lysosome | PEX19 | peroxisome |
| PEX14 | peroxisome | PEX11B | peroxisome |
| PEX19 | peroxisome | PEX16 | peroxisome |
| PEX19 | peroxisome | PEX3 | peroxisome |
| PEX19 | peroxisome | PPP3R1 | cytosol, plasma |
| PPP3CA | nucleus, cytosol, | | membrane |
| | plasma membrane | | (anchored), |
| | (peripheral) | | nucleus |
| PRKAR2A | plasma membrane, | EZR | plasma membrane |
| | cytoplasm, nucleus | | (peripheral), |
| PSMD4 | cytosol, nucleus | | cytoskeleton, nucleus, |
| PTK2 | plasma membrane | | cytosol, endosome |
| | (associated), | RAD23A | nucleus, cytosol |
| | cytoskeleton, nucleus, | SRC | plasma membrane, |
| | cytosol | | cytoskeleton, |
| PTPN11 | nucleus, cytoplasm | | cytoplasm, nucleus, |
| RAC1 | plasma membrane | | mitochondrion |
| | (anchored), cytoplasm | FRS2 | plasma membrane, |
| RAF1 | cytosol, plasma | | cytosol, endosome |
| | membrane, nucleus | ARFIP2 | plasma membrane, |
| RCC1 | nucleus | | cytosol, |
| RET | plasma membrane, | | Golgi apparatus |
| | endosome | RAP1A | plasma membrane |
| RHOA | cytoskeleton, plasma | | (anchored), endosome |
| | membrane (anchored) | RAN | nucleus, cytosol |
| RIPK2 | cytoplasm | FRS2 | plasma membrane, |
| RPA2 | nucleus | | cytosol, endosome |
| S100A1 | nucleus, cytoplasm | ARHGAP1 | cytoplasm |
| S100A6 | plasma membrane | NOD1 | plasma membrane, |
| | (peripheral), nucleus, | | cytoplasm |
| | cytoplasm | RPA3 | nucleus |
| SKP1 | cytoplasm, nucleus | S100B | nucleus, cytoplasm |
| SKP1 | cytoplasm, nucleus | S100B | nucleus, cytoplasm |
| SMAD1 | nucleus, cytoplasm | BTRC | nucleus, cytosol |
| SMAD3 | nucleus, cytoplasm | SKP2 | nucleus, cytoplasm |
| SMAD4 | nucleus, cytoplasm | SMAD4 | nucleus, cytoplasm |
| TNFSF10 | extracellular, plasma | SMAD4 | nucleus, cytoplasm |
| | membrane | DCP1A | nucleus |
| TP53 | nucleus, | TNFRSF10B | plasma membrane |
| | mitochondrion, | UBE2I | nucleus, cytosol |
| | ER | CASP3 | cytoplasm, nucleus |
| XIAP | nucleus, cytoplasm | CASP7 | cytoplasm, nucleus |
| XIAP | nucleus, cytoplasm | CASP9 | cytosol, |
| XIAP | nucleus, cytoplasm | | mitochondrion |

Note:
the “Prey” column should be read next to the “Bait” column.

TABLE 2
(b) Random reference set

| bait | | prey | |
|---|---|---|---|
| | subcellular | | subcellular |
| name | localization | name | localization |
| AKT1 | cell membrane | PEX19 | peroxisome |
| | (associated), | SKP2 | nucleus, cytoplasm |
| | cytoplasm, nucleus | LMNB1 | nucleus |
| ARF1 | Golgi apparatus | PEX16 | peroxisome |
| | (anchored), plasma | RAD23A | nucleus, cytosol |
| | membrane (anchored) | TP53 | nucleus, |
| ATF3 | nucleus | | mitochondrion, |
| B2M | extracellular | | ER |
| B2M | extracellular | DCP1A | nucleus |
| B2M | extracellular | RAP1A | plasma membrane |
| BAD | mitochondrion | | (anchored), endosome |
| | (outer membrane), | MAD1L1 | cytoskeleton, nucleus, |
| | cytoplasm | | kinetochore |
| BAK1 | mitochondrion | MCM5 | nucleus, cytosol |
| | (outer membrane) | VAV1 | cytosol, plasma |
| BDNF | extracellular | | membrane |
| BDNF | extracellular | BCL2L1 | mitochondrion, |
| CASP2 | cytosol, | | cytoskeleton, nucleus, |
| | mitochondrion, | | cytosol |
| | nucleus | GTF2F2 | nucleus |
| CBLB | cytoplasm | HGS | endosome, cytoplasm |
| CD2 | cell membrane | DDIT3 | nucleus |
| CD2 | cell membrane | PEX11B | peroxisome |
| CDK2 | nucleus, cytoplasm, | DRAP1 | nucleus |
| | cytoskeleton, | ARHGAP1 | cytoplasm |
| | endosome | CASP9 | cytosol, |
| CDKN1A | nucleus | | mitochondrion |
| CDKN1B | nucleus, endosome | CD58 | plasma membrane |
| CEBPG | nucleus | MCM3 | nucleus |
| CEBPG | nucleus | NCBP2 | nucleus |
| CGA | extracellular | UBE2I | nucleus, cytosol |
| CGA | extracellular | MAP2K5 | nucleus, cytoplasm |
| CGA | extracellular | ORC4 | nucleus |
| CRK | cytoplasm, nucleus, | SMAD4 | nucleus, cytoplasm |
| | membrane (associated) | NRG1 | membrane, |
| CXCL1 | extracellular | | extracellular |
| CXCL1 | extracellular | NTF4 | extracellular |
| CXCLI | extracellular | DDIT3 | nucleus |
| DDIT3 | nucleus | PCNA | nucleus |
| DR1 | nucleus | PTK2 | plasma membrane |
| ERBB3 | plasma membrane | | (associated), |
| ERBB3 | plasma membrane | | cytoskeleton, nucleus, |
| FABP5 | nucleus, cytosol, | | cytosol |
| | membrane, | GRAP2 | endosome, cytosol, |
| | extracellular | | nucleus |
| FEN1 | nucleus | NCK1 | plasma membrane, |
| FGF1 | extracellular, cytosol, | | ER, nucleus, cytosol |
| | nucleus | RB1 | nucleus |
| FGF1 | extracellular, cytosol, | FRS2 | plasma membrane, |
| | nucleus | | cytosol, endosome |
| GADD45A | nucleus | PDPK1 | plasma membrane |
| GADD45A | nucleus | | (peripheral), nucleus, |
| GRAP2 | endosome, cytosol, | | cytosol |
| | nucleus | NFE2L1 | ER, nucleus |
| GRB2 | plasma membrane, | PEX3 | peroxisome |
| | cytoplasm, nucleus | NOD1 | plasma membrane, |
| GTF2F1 | nucleus | | cytoplasm |
| HBA2 | cytosol | CKS1B | nucleus |
| HDAC1 | nucleus | LAT | plasma membrane |
| HDAC1 | nucleus | PPP3R1 | cytosol, plasma |
| HIF1A | nucleus, cytosol | | membrane |
| IFIT1 | cytoplasm | | (anchored), |
| IGF2 | extracellular | | nucleus |
| IGF2 | extracellular | ARFIP2 | plasma membrane, |
| IGF2 | extracellular | | cytosol, Golgi |
| JUNB | nucleus | | apparatus |
| LCP2 | cytoplasm | RELA | nucleus, cytoplasm |
| LGALS3 | extracellular, plasma | ARFIP2 | plasma membrane, |
| | membrane, nucleus, | | cytosol, Golgi |
| | cytoplasm | | apparatus |
| LMNA | nucleus | HSP90AA1 | plasma membrane, |
| LSM3 | nucleus | | cytoplasm, nucleus |
| LSM3 | nucleus | RPA3 | nucleus |
| MAD2L1 | nucleus, cytoskeleton, | PTPN11 | nucleus, cytoplasm |
| | kinetochore | CRADD | nucleus, cytosol |
| MAD2L1 | nucleus, cytoskeleton, | LSM2 | nucleus |
| | kinetochore | PEX3 | peroxisome |
| MAFG | nucleus | FGFR1 | plasma membrane |
| MAFG | nucleus | LGALS3BP | extracellular, nucleus, |
| MAPK7 | nucleus, cytoplasm | | plasma membrane |
| MCM2 | nucleus | NFE2L1 | ER, nucleus |
| NCBP1 | nucleus | NTF4 | extracellular |
| NF2 | plasma membrane | PEX11B | peroxisome |
| | (associated), nucleus | TNFRSF10B | plasma membrane |
| NR3C1 | mitochondrion, | PEX16 | peroxisome |
| | cytosol, nucleus, | S100B | nucleus, cytoplasm |
| | cytoskeleton | S100B | nucleus, cytoplasm |
| ORC2 | nucleus | FOS | nucleus, ER, cytosol |
| ORC2 | nucleus | RAD23A | nucleus, cytosol |
| PDE4D | plasma membrane | HBB | cytosol |
| | (associated), | UBE2I | nucleus, cytosol |
| | cytoskeleton | EZR | plasma membrane |
| PDGFRB | plasma membrane, | | (peripheral), |
| | lysosome | | cytoskeleton, |
| PEX14 | peroxisome | | nucleus, cytosol, |
| PEX19 | peroxisome | | endosome |
| PPP3CA | nucleus, cytosol, | RAN | nucleus, cytosol |
| | plasma membrane | NRG1 | membrane, |
| | (peripheral) | | extracellular |
| PRKAR2A | plasma membrane, | BATF | nucleus |
| | cytoplasm, nucleus | FOS | nucleus, ER, cytosol |
| PSMD4 | cytosol, nucleus | SRC | plasma membrane, |
| PSMD4 | cytosol, nucleus | | cytoskeleton, |
| RAC1 | plasma membrane | | cytoplasm, nucleus, |
| | (anchored), cytoplasm | | mitochondrion |
| RCC1 | nucleus | NOD1 | plasma membrane, |
| RET | plasma membrane, | | cytoplasm |
| | endosome | TCL1A | nucleus, ER |
| RHOA | cytoskeleton, plasma | BTRC | nucleus, cytosol |
| | membrane (anchored) | CASP7 | cytoplasm, nucleus |
| RHOA | cytoskeleton, plasma | NCBP2 | nucleus |
| | membrane (anchored) | CCNA1 | nucleus |
| RIPK2 | cytoplasm | ZBTB16 | nucleus |
| RIPK2 | cytoplasm | IGFBP4 | extracellular |
| RPA2 | nucleus | PDGFR8 | plasma membrane, |
| S100A1 | nucleus, cytoplasm | | lysosome |
| S100A6 | plasma membrane | S100A7 | extracellular, |
| | (peripheral), nucleus, | | cytoplasm, nucleus |
| | cytoplasm | CASP3 | cytoplasm, nucleus |
| SKP1 | cytoplasm, nucleus | RAN | nucleus, cytosol |
| SMAD1 | nucleus, cytoplasm | CXCR2 | plasma membrane |
| SMAD3 | nucleus, cytoplasm | GRB2 | plasma membrane, |
| SMAD4 | nucleus, cytoplasm | | cytoplasm, nucleus |
| TNFSF10 | extracellular, plasma | CXCR2 | plasma membrane |
| | membrane | LAT | plasma membrane |
| XIAP | nucleus, cytoplasm | CDK6 | nucleus, cytoskeleton, |
| | | | cytosol |
| | | ARFIP2 | plasma membrane, |
| | | | cytosol, Golgi |
| | | | apparatus |

TABLE 3
Detection of reference sets with SIMPL
(b) Detection with SIMPL in bait-IN/prey-CIC format

| bait | prey | reference set | PLU_mean | PLU_sd | SIMPL result |
|---|---|---|---|---|---|
| DR1 | DRAP1 | | 3.969868 | 0.473734 | positive |
| GTF2F1 | GTF2F2 | | 3.240792 | 0.379569 | positive |
| LSM3 | LSM2 | | 3.034348 | 0.299742 | positive |
| CCND3 | CDK6 | | 2.724104 | 0.493514 | positive |
| RPA2 | RPA3 | | 2.722426 | 0.237577 | positive |
| CBLB | GRB2 | | 2.612294 | 0.620953 | positive |
| MAD2L1 | MAD1L1 | | 2.51995 | 0.268931 | positive |
| CASP2 | CRADD | | 2.448596 | 1.268555 | positive |
| PEX14 | PEX19 | | 2.167057 | 0.005568 | positive |
| NCBP1 | NCBP2 | | 2.146787 | 0.111236 | positive |
| JUNB | BATF | | 2.038777 | 0.198681 | positive |
| CDK2 | CKS1B | | 1.882465 | 0.186788 | positive |
| LCP2 | GRAP2 | | 1.860639 | 0.213948 | positive |
| BAD | BCL2L1 | | 1.852165 | 0.24557 | positive |
| BAK1 | BCL2L1 | | 1.783491 | 0.097307 | positive |
| PEX19 | PEX3 | | 1.769572 | 0.165381 | positive |
| MAFG | NFE2L1 | | 1.717193 | 0.115782 | positive |
| PPP3CA | PPP3R1 | | 1.678497 | 0.161701 | positive |
| ATF3 | DDIT3 | | 1.637576 | 0.14194 | positive |
| GRB2 | VAV1 | | 1.388383 | 0.138295 | positive |
| HBA2 | HBB | | 1.36129 | 0.270433 | positive |
| SKP1A | BTRC | | 1.255416 | 0.139085 | positive |
| ERBB3 | NRG1 | | 1.219217 | 0.410585 | positive |
| LMNA | RB1 | | 1.183392 | 0.995994 | positive |
| PEX19 | PEX11B | | 1.179537 | 0.206526 | positive |
| TNFSF10 | TNFRSF10B | | 1.177707 | 0.062024 | positive |
| RIPK2 | NOD1 | | 1.173035 | 0.16047 | positive |
| PEX19 | PEX16 | | 1.091572 | 0.144247 | positive |
| RET | NCBP2 | | 1.060548 | 0.97354 | positive |
| HIF1A | TP53 | | 1.016036 | 0.093678 | positive |
| CD2 | CD58 | | 0.972098 | 0.086792 | positive |
| MAPK7 | MAP2K5 | | 0.948185 | 0.093095 | positive |
| IGF2 | IGFBP4 | | 0.943004 | 0.19349 | positive |
| MCM2 | MCM3 | | 0.938011 | 0.061255 | positive |
| PSMD4 | RAD23A | | 0.91937 | 0.131715 | positive |
| RIPK2 | PDGFRB | | 0.881284 | 0.139867 | positive |
| LMNA | LMNB1 | | 0.861232 | 0.12271 | positive |
| PSMD4 | NOD1 | | 0.845249 | 0.30456 | positive |
| NF2 | HGS | | 0.835999 | 0.04363 | positive |
| S100A1 | S100B | | 0.822118 | 0.046727 | positive |
| XIAP | CASP9 | | 0.810968 | 0.041818 | positive |
| LCP2 | VAV1 | | 0.78949 | 0.111015 | positive |
| CEBPG | FOS | | 0.788308 | 0.340615 | positive |
| RCC1 | RAN | | 0.747932 | 0.095913 | positive |
| GRB2 | PTK2 | | 0.740728 | 0.02354 | positive |
| CASP2 | VAV1 | | 0.69552 | 0.123926 | positive |
| RHOA | ZBTB16 | | 0.690993 | 0.13011 | positive |
| CDKN1B | CCNA1 | | 0.68555 | 0.01238 | positive |
| IFIT1 | EIF3E | | 0.675818 | 0.297155 | positive |
| RAC1 | ARFIP2 | | 0.672191 | 0.186376 | positive |
| LCP2 | NCK1 | | 0.661524 | 0.061033 | positive |
| NR3C1 | HSP90AA1 | | 0.652489 | 0.163939 | positive |
| DDIT3 | FOS | | 0.64182 | 0.090846 | positive |
| PDE4D | RACK1 | | 0.638768 | 0.074738 | positive |
| NR3C1 | RELA | | 0.630063 | 0.089936 | positive |
| BDNF | NTF4 | | 0.628784 | 0.045742 | positive |
| RET | FRS2 | | 0.608223 | 0.111882 | positive |
| PTK2/FAK | SRC | | 0.598863 | 0.074534 | positive |
| RAF1 | RAP1A | | 0.577907 | 0.230264 | positive |
| BAK1 | RAP1A | | 0.564385 | 0.033061 | negative |
| RIPK2 | IGFBP4 | | 0.555123 | 0.097446 | negative |
| HDAC1 | RB1 | | 0.539814 | 0.137497 | negative |
| PDGFRB | PTPN11 | | 0.53935 | 0.150515 | negative |
| IGF2 | RPA3 | | 0.524501 | 0.07459 | negative |
| CDKN1A | CCNA1 | | 0.516253 | 0.079258 | negative |
| BAD | DCP1A | | 0.49879 | 0.053441 | negative |
| HDAC1 | ZBTB16 | | 0.491878 | 0.02719 | negative |
| ARF1 | ARFIP2 | | 0.491174 | 0.074615 | negative |
| AKT1 | PDPK1 | | 0.486209 | 0.068759 | negative |
| RHOA | ARHGAP1 | | 0.485965 | 0.023812 | negative |
| CEBPG | CASP9 | | 0.481506 | 0.108028 | negative |
| NF2 | FOS | | 0.480537 | 0.188662 | negative |
| PTPN11 | FRS2 | | 0.474205 | 0.206277 | negative |
| MAFG | TNFRSF10B | | 0.458591 | 0.030775 | negative |
| XIAP | CASP7 | | 0.451877 | 0.032686 | negative |
| XIAP | ARFIP2 | | 0.439407 | 0.055467 | negative |
| CGA | CGB5 | | 0.435412 | 0.070967 | negative |
| ORC2 | MCM10 | | 0.429797 | 0.002876 | negative |
| IGF2 | HSP90AA1 | | 0.403788 | 0.193402 | negative |
| SMAD3 | SMAD4 | | 0.398739 | 0.155898 | negative |
| SMAD4 | DCP1A | | 0.398168 | 0.049793 | negative |
| B2M | HLA-C | | 0.38573 | 0.035579 | negative |
| SKP1A | SKP2 | | 0.375745 | 0.047265 | negative |
| FGF1 | NCK1 | | 0.373869 | 0.057335 | negative |
| RHOA | CCNA1 | | 0.359663 | 0.295578 | negative |
| MCM2 | MCM5 | | 0.349833 | 0.016165 | negative |
| FEN1 | PCNA | | 0.34944 | 0.020362 | negative |
| JUNB | PTPN11 | | 0.345289 | 0.003788 | negative |
| FEN1 | GRAP2 | | 0.341864 | 0.020429 | negative |
| MAFG | PEX11B | | 0.341491 | 0.075287 | negative |
| FGF1 | FGFR1 | | 0.334291 | 0.103549 | negative |
| IFIT1 | RELA | | 0.327506 | 0.089449 | negative |
| FGF1 | RB1 | | 0.324257 | 0.070172 | negative |
| HIF1A | ARFIP2 | | 0.32423 | 0.154696 | negative |
| TNFSF10 | CDK6 | | 0.316067 | 0.047023 | negative |
| XIAP | CASP3 | | 0.315704 | 0.101024 | negative |
| PEX14 | NRG1 | | 0.309753 | 0.021418 | negative |
| B2M | HLA-B | | 0.305548 | 0.004724 | negative |
| ARF1 | SKP2 | | 0.30118 | 0.046346 | negative |
| CD2 | GTF2F2 | | 0.298631 | 0.033051 | negative |
| ORC2 | ORC4 | | 0.297486 | 0.067726 | negative |
| B2M | HLA-A | | 0.288261 | 0.005242 | negative |
| ERBB3 | DDIT3 | | 0.279722 | 0.080593 | negative |
| CDK2 | DDIT3 | | 0.275186 | 0.028277 | negative |
| CBLB | BCL2L1 | | 0.272223 | 0.093711 | negative |
| LSM3 | FGFR1 | | 0.272014 | 0.024866 | negative |
| CXCL1 | MAP2K5 | | 0.269603 | 0.039369 | negative |
| IGF2 | ARFIP2 | | 0.265551 | 0.209521 | negative |
| GADD45A | PDPK1 | | 0.264868 | 0.032061 | negative |
| CRK | PDGFRB | | 0.260567 | 0.012792 | negative |
| RAC1 | BTRC | | 0.256375 | 0.024785 | negative |
| CD2 | HGS | | 0.253155 | 0.023991 | negative |
| LGALS3 | LGALS3BP | | 0.247573 | 0.036982 | negative |
| HDAC1 | PPP3R1 | | 0.247043 | 0.123296 | negative |
| PPP3CA | FOS | | 0.245191 | 0.063541 | negative |
| MAPK7 | PEX16 | | 0.230529 | 0.116863 | negative |
| CDKN1B | DRAP1 | | 0.228777 | 0.013117 | negative |
| ORC2 | HBB | | 0.227103 | 0.036994 | negative |
| LMNA | PEX3 | | 0.224467 | 0.085672 | negative |
| GRB2 | LAT | | 0.222832 | 0.040559 | negative |
| HDAC1 | LAT | | 0.221675 | 0.04251 | negative |
| BDNF | MCM5 | | 0.220349 | 0.037035 | negative |
| AKT1 | PEX19 | | 0.218458 | 0.169793 | negative |
| S100A6 | S100B | | 0.217399 | 0.079923 | negative |
| NCBP1 | S100B | | 0.216886 | 0.035444 | negative |
| SMAD4 | LAT | | 0.214672 | 0.033353 | negative |
| GADD45A | FRS2 | | 0.210346 | 0.029615 | negative |
| GRAP2 | LAT | | 0.194509 | 0.017824 | negative |
| TP53 | UBE2I | | 0.194156 | 0.04363 | negative |
| LCP2 | CRADD | | 0.193135 | 0.052165 | negative |
| BDNF | MAD1L1 | | 0.190383 | 0.031383 | negative |
| PRKAR2A | SRC | | 0.18566 | 0.032192 | negative |
| NR3C1 | RAD23A | | 0.18329 | 0.111034 | negative |
| SMAD1 | SMAD4 | | 0.181746 | 0.035855 | negative |
| CXCL1 | CXCR2 | | 0.177645 | 0.013534 | negative |
| FABP5 | PTK2 | | 0.173788 | 0.014644 | negative |
| HBA2 | CKS1B | | 0.170859 | 0.01745 | negative |
| LGALS3 | LSM2 | | 0.162419 | 0.009571 | negative |
| S100A1 | CASP3 | | 0.15799 | 0.023719 | negative |
| PEX19 | BATF | | 0.153584 | 0.025886 | negative |
| CRK | UBE2I | | 0.152743 | 0.007107 | negative |
| SMAD1 | GRB2 | | 0.15124 | 0.022569 | negative |
| SMAD3 | CXCR2 | | 0.143507 | 0.00956 | negative |
| PDGFRB | RAN | | 0.143002 | 0.019113 | negative |
| PDE4D | EZR | | 0.136674 | 0.002281 | negative |
| ATF3 | LMNB1 | | 0.134442 | 0.017108 | negative |
| FABP5 | S100A7 | | 0.13371 | 0.037526 | negative |
| MAD2L1 | NTF4 | | 0.13325 | 0.019956 | negative |
| GTF2F1 | NOD1 | | 0.132609 | 0.005166 | negative |
| GRAP2 | NFE2L1 | | 0.131061 | 0.026992 | negative |
| CGA | CD58 | | 0.130693 | 0.00792 | negative |
| LSM3 | LGALS3BP | | 0.12927 | 0.003416 | negative |
| RCC1 | CASP7 | | 0.124715 | 0.052199 | negative |
| PRKAR2A | EZR | | 0.123433 | 0.038166 | negative |
| ORC2 | UBE2I | | 0.118785 | 0.027584 | negative |
| CDKN1A | PEX11B | | 0.113346 | 0.068613 | negative |
| AKT1 | TCL1A | | 0.110536 | 0.013041 | negative |
| PSMD4 | TCL1A | | 0.108356 | 0.009092 | negative |
| SKP1A | CXCR2 | | 0.106803 | 0.014761 | negative |
| S100A6 | RAN | | 0.105488 | 0.018279 | negative |
| CXCL1 | ORC4 | | 0.10321 | 0.025572 | negative |
| MCM2 | S100B | | 0.101573 | 0.012127 | negative |
| CXCL1 | SMAD4 | | 0.101152 | 0.019587 | negative |
| DDIT3 | NRG1 | | 0.096285 | 0.034975 | negative |
| MAD2L1 | NFE2L1 | | 0.081861 | 0.002436 | negative |
| ERBB3 | PCNA | | 0.081193 | 0.027165 | negative |
| CGA | MCM3 | | 0.073756 | 0.018602 | negative |
| GADD45A | PCNA | | 0.061656 | 0.002698 | negative |
| RPA2 | S100A7 | | 0.060628 | 0.003192 | negative |
| GRB2 | PEX3 | | 0.058757 | 0.026112 | negative |
| CEBPG | ARHGAP1 | | 0.058463 | 0.025787 | negative |
| DR1 | NTF4 | | 0.052933 | 0.007415 | negative |
| B2M | PEX16 | | 0.040836 | 0.010143 | negative |
| CGA | NCBP2 | | 0.036345 | 0.006647 | negative |
| B2M | TP53 | | 0.032948 | 0.007287 | negative |
| B2M | RAD23A | | 0.015545 | 0.002994 | negative |

TABLE 4
Mitochondrial PPIs tested by SIMPL
Bait

| Name | Alias | Transit signal | Localization |
|---|---|---|---|
| SDHA | SDH1, SDHF | N-terminal | Matrix protein peripheral to inner membrane |
| ETFA | alpha-ETF | N-terminal | Matrix |
| ETFA | alpha-ETF | N-terminal | Matrix |
| TIMM10 | TIM10 | middle | Intermembrane space |
| CHCHD6 | MIC25 | unknown | Lipid-anchored to inner membrane |
| | | | in the intermembrane space. |
| UQCRC2 | QCR2, UQCR2 | N-terminal | Matrix side peripheral protein to inner |
| | | | membrane |
| TIMM50 | TIM50, TIM50L | N-terminal | Transmembrane protein at the inner |
| | | | mitochondrial membrane, exposing the |
| | | | C-terminus to the intermembrane space with |
| | | | interacting with TIMM23 N-terminal domain |
| ATP5MC1 | ATP5G1, ATP5A, ATP5G | N-terminal | Multipass transmembrane protein in the inner |
| | | | membrane with both N- and C-termini in the |
| | | | intermembrane space |
| NDUFV1 | UQOR1, CI51KD | N-terminal | Matrix protein peripheral to inner |
| | | | membrane |
| PDHA1 | PHE1A, PDHA, PDHAD | N-terminal | Matrix |

| Prey | | | | | |
|---|---|---|---|---|---|
| Name | Alias | Transit signal | Localization | Function | Reference |
| SDHB | SDH2 | N terminal | Matrix protein peripheral | complex II | Sun et al. Cell (2005) |
| | | | to inner membrane | | 121, 1043-1057 |
| ETFB | beta-ETF | unknown | Matrix | ETF complex, | Toogood et al, |
| | | | | OXPHOS | J Biol Chem (2004) |
| | | | | complexes | 279, 32904-32912 |
| ETFRF1 | LYRM5 | unknown | Matrix | ETF complex, | Floyd et al, Mol Cell |
| | | | | OXPHOS | (2016) 63, 621-632 |
| | | | | complexes | |
| TIMM9 | TIM9 | middle | Intermembrane space | TIM9/10 complex. | Webb et al, Mol Cell |
| | | | | | (2006) 21. 123-133 |
| CHCHD3 | MIC19 | unknown | Lipid-anchored to inner | MICOS complex | Kozjak-Pavlovic, |
| | | | membrane in the | | Cell Tissue |
| | | | intermembrane space. | | Res (2017) 367: 63-93 |
| UQCRC2 | QCR2, | N-terminal | Matrix side peripheral | complex III | Guo et al, Cell |
| | UQCR2 | | protein to inner membrane | | (2017) 170, 1247-1257 |
| TIMM23 | TIM23 | unknown | Inner membrane multiple | TIM23 complex | Geissler et al, Cell |
| | | | transmembrane protein | | (2002) 111, 507-518; |
| | | | with N-terminus in the | | Yamamoto et al, Cell |
| | | | intermembrane space | | 111 (2002), 519-528; |
| | | | | | Demishtein-Zohary |
| | | | | | et al, Cell Tissue |
| | | | | | Res (2017) 367, 33-41 |
| ATP5MC1 | ATP5G1, | N-terminal | Multipass transmembrane | Fo complex of | Rastogi et al, Nature |
| | ATP5A, | | protein in the inner | ATPase | (1999) 402. 263-268 |
| | ATP5G | | membrane with both N- | | |
| | | | and C-termini in the | | |
| | | | intermembrane space | | |
| NDUFV3 | CI9KD | N-terminal | Matrix protein peripheral | N module of | Guo et al, Cell (2017) |
| | | | to inner membrane | complex I | 170, 1247-1257 |
| PDHB | PHE1B, | N-terminal | Matrix | Pyruvate | Ciszak et al, |
| | PDHE1B, | | | dehydrogenase | J Biol Chem (2003) |
| | PDHBD | | | complex | 278, 21240-21246 |

TABLE 5
C. elegans references set studied by SIMPL

| | Reference | Bait Protein (IN-V5/HA) | | Prey Protein (IC/CIC-FLAG) | | Configuration | | ELISA Value Replicate 1 | | ELISA Value Replicate 2 | | | | |
|---|---|---|---|---|---|---|---|---|---|---|---|---|---|---|
| # | Set | CDS | Protein | CDS | Protein | IN/IC | IC/CIC | IN/IC | IC/CIC | IN/IC | IC/CIC | PPI Description/Homologs | Source | Pubmed ID |
| 1 | Positive | T05H4.2 | FBXA-196 | F46A9.4 | SKR-2 | Yes | Yes | 0.81 | 1.1 | 0.51 | 0.28 | SKP1/F-Dox Domain | CePRS | |
| 2 | Positive | Y45F10C.3 | FBXA-215 | F46A9.4 | SKR-2 | Yes | Yes | 0.84 | 0.73 | 0.58 | 0.42 | SKP1/F-Box Domain | CePRS | |
| 3 | Positive | ZK792.6 | LET 60 | AC7.2 | SOC 2 | Yes | Yes | 1.2 | 0.79 | 0.59 | 0.70 | SHOC2/HRAS | CePRS | |
| 4 | Positive | F53F10.5 | NPP-11 | K07F5.13 | NPP-1 | Yes | inj. | 1.8 | — | 0.92 | — | Nuclear pore complex components Nup54 | LIT/EE | 7531196 |
| | | | | | | | | | | | | and Nup62 | | |
| 5 | Positive | F58F6.4 | RFC-2 | F44B9.8 | F44B9.8 | Yes | inj. | 0.66 | — | 0.88 | — | RFC5/RFC2 Replication factor subunits | LIT/EE | 15201901 |
| 6 | Positive | T10E9.1 | T10E9.1 | F46A9.4 | SKR-2 | Yes | inj. | 0.78 | — | 0.55 | — | SKP1/F-Box Domain | CePRS | |
| 7 | Positive | T05G5.3 | CDK-1 | ZC168.4 | CYB-1 | inj. | Yes | — | 1.1 | — | 0.80 | Cyclin B2/Cyclin Dependent Kinase 1 | LIT/EE | 7575488 |
| 8 | Positive | C06G3.10 | COGC-2 | Y51H7C.6 | COGC-4 | n.c. | n.c. | — | — | — | — | COG2/COG4 (component of oligomeric | LIT/EE | 15047703 |
| | | | | | | | | | | | | golgi complex 4) | | |
| 9 | Positive | F10B5.6 | EMB-27 | B0511.9 | CDC-26 | inj. | imj. | — | — | — | — | Cdc26p and Cdc16p components of the | LIT/EE | 8895471 |
| | | | | | | | | | | | | anaphase promoting complex | | |
| 10 | Positive | K08B4.1 | LAG-1 | C32A3.1 | SEL-8 | n.c. | n.c. | — | — | — | — | CSL (CBF-1, Su(H), Lag-1)/Mastermind | CePES | |
| 11 | Positive | F58A3.1 | LDB-1 | F46C8.5 | CEH-14 | n.c | n.c | — | — | — | — | LDB2 (LIM domain binding 2)/LHX3 | CePRS | |
| | | | | | | | | | | | | (LIM homeobox 3) and LHX4 (LIM | | |
| | | | | | | | | | | | | homeobox 4) | | |
| 12 | Positive | M7.1 | LET-70 | F54G8.4 | NHL-1 | n.c | n.c. | — | — | — | — | UBE2D2 (ubiquitin conjugating enzyme | CePRS | |
| | | | | | | | | | | | | E2 D2)/TRIM2 (tripartite motif | | |
| | | | | | | | | | | | | containing 2) and TRIM3 (tripartite | | |
| | | | | | | | | | | | | motif containing 3) | | |
| 13 | Positive | F59E12.5 | NPL-4.2 | F19B6.2 | UFD-1 | n.c | n.c. | — | — | — | — | NPLOC4 (NPL4 homolog, ubiquitin | LIT/EE | 10811609 |
| | | | | | | | | | | | | recognition factor)/UFD1 (ibiquitin | | |
| | | | | | | | | | | | | recognition factor in ER associated | | |
| | | | | | | | | | | | | degradation 1) | | |
| 14 | Positive | F59A2.1 | KPP-9 | K01G5.4 | RAN-1 | n.c. | n.c. | — | — | — | — | RANBP1 (RAN binding protein 1)/RAN | LIT/EE | 7603572 |
| | | | | | | | | | | | | (RAN, member RAS oncogene family) | | |
| 15 | Positive | F21C3.4 | RDE-2 | F21C3.4 | RDE-2 | n.c. | n.c. | — | — | — | — | RDE2 RNA interference protein | CePRS | |
| 16 | Positive | M03D4.1 | ZEN-4 | B0207.4 | AIR-2 | inj. | inj. | — | — | — | — | Aurora Kinase C/KIF23 | CePRS | |
| 17 | Positive | Y37H2A.5 | FBXA-210 | F46S9.4 | SKR-2 | inj. | inj. | — | — | — | — | SKP1/F-Box Domain | CePRS | |
| 18 | Positive | Y113G7B.5 | FOG-2 | T23G11.3 | GLD-1 | inj. | inj. | — | — | — | — | QKI (QKI, KH domain containing RNA | CePRS | |
| | | | | | | | | | | | | binding)/Germline protein | | |
| 19 | Positive | M7.1 | LET-70 | F16A11.1 | F16A11.1 | n.c. | n.c. | — | — | — | — | UBE2D2 (ubiquitin conjugating enzyme | CePRS | |
| | | | | | | | | | | | | E2 D2)/RSPRY1 (ring finger and | | |
| | | | | | | | | | | | | SPRY domain containing 1 | | |
| 20 | Positive | M7.1 | LET-70 | C45G7.4 | C45G7.4 | n.c | n.c. | — | — | — | — | UBE2D2 (ubiquitin conjugating enzyme | CePRS | |
| | | | | | | | | | | | | E2 D2)/TRIM13 | | |
| 21 | Positive | ZK1098.8 | MUT-7 | F21C3.4 | RDE-2 | n.c. | n,c. | — | — | — | — | EXD3 (exonuclease 3′-5′ domain | CePRS | |
| | | | | | | | | | | | | containing 3)/RDE2 involved in RNA | | |
| | | | | | | | | | | | | interference | | |
| 22 | Positive | R06F6.5 | NPP-19 | Y37E3.15 | NPP-15 | inj. | inj. | — | — | — | — | Nuclear pore complex components | LIT/EE | 16631361 |
| | | | | | | | | | | | | Nup35 and Nup93 | | |
| 23 | Positive | Y49E10.14 | PIE-1 | F59B2.6 | ZIF-1 | inj. | inj. | — | — | — | — | mRNA 3′-UTR binding activity/ubiquitin- | CePRS | |
| | | | | | | | | | | | | dependent protein | | |
| 24 | Positive | T27F2.1 | SKP-1 | K08B4.1 | LAG-1 | n.c | n.c. | — | — | — | — | SNW1 (SNCS1 (CBF-1, Su(H), Lag-1)W | CePRS | |
| | | | | | | | | | | | | domain containing 1)/ | | |
| 25 | Positive | T07E3.4 | T07E3.4 | F46A9.4 | SKR-2 | inj. | inj. | — | — | — | — | SKP1/F-Box Domain | CePRS | |
| 26 | Positive | Y39G10A.12 | TPXL-1 | K07C11.2 | AIR-1 | inj. | inj. | — | — | — | — | Interaction between Aurora A kinase | LIT/EE | 16054030 |
| | | | | | | | | | | | | and microtubule binding protein TPX2 | | |
| 27 | Positive | Y54E5B.4 | UBC-16 | F54G8.4 | NHL-1 | n.c. | n.c | — | — | — | — | BBE2W (ubiquitin conjugating enzyme | CePRS | |
| | | | | | | | | | | | | E2 W)/TRIM2 (tripartite motif containing 2) | | |
| | | | | | | | | | | | | and TRIM3 (tripartite motif containing 3) | | |

Key to Abbreviations
Yes = Line successfully established and analyzed by SIMPL ELISA platform
n.c. = not cloned/cloning unsuccessful
inj. = injected, but no transgenic line could be established with expression of both Bait and Prey
CePRS = C. elegans Positive Reference Set
LIT/EE = Literature derived and previously confirmed by yeast two-hybrid.
Description/Homolog information from WormBase

SEQUENCE LISTING

GP41-1 IN (WT) (SEQ ID NO: 1)
CLDLKTQVQT PQGMKEISNI QVGDLVLSNT GYNEVLNVFP
KSKKKSYKIT LEDGKEIICS EEHLFPTQTG EMNISGGLKE
GMCLYVKE
GP41-1 IC (WT) (SEQ ID NO: 2)
MMLKKILKIE ELDERELIDI EVSGNHLFYA NDILTHN
GP41-1 IN (C25) (SEQ ID NO: 3)
CLDLKTQVQT PQGMKEISNI QVGDLVLSNT GYNEVLNVFP
KSKKKSYKIT LEDGKEIICS EEHLFPTQTG EMNISGGLKE
GMCLYVKEMM LKKILKIEEL
GP41-1 IC (C25) (SEQ ID NO: 4)
DERELIDIEV SGNHLFYAND ILTHN

### REFERENCES

1. Yao, Z., Petschnigg, J., Ketteler, R. & Stagljar, I. Application guide for omics approaches to cell signaling. Nat. Chem. Biol. 11, 387-397 (2015).
2. Petschnigg, J., Snider, J. & Stagljar, I. Interactive proteomics research technologies: recent applications and advances. Curr. Opin. Biotechnol. 22, 50-58 (2011).
3. Snider, J. et al. Fundamentals of protein interaction network mapping. Mol. Syst. Biol. 11, 848-848 (2015).
4. Suter, B., Kittanakom, S. & Stagljar, I. Two-hybrid technologies in proteomics research. Curr. Opin. Biotechnol. 19, 316-323 (2008).
5. Gogarten, J. P., Senejani, A. G., Zhaxybayeva, O., Olendzenski, L. & Hilario, E. Inteins: Structure, Function, and Evolution. Annu. Rev. Microbiol. 56, 263-287 (2002).
6. Shah, N. H. & Muir, T. W. Inteins: nature's gift to protein chemists. Chem. Sci. 5, 446-461 (2014).
7. Aranko, A. S., Wlodawer, A. & Iwaï, H. Nature's recipe for splitting inteins. Protein Eng. Des. Sel. 27, 263-271 (2014).
8. Wood, D. W. & Camarero, J. A. Intein applications: From protein purification and labeling to metabolic control methods. J. Biol. Chem. 289, 14512-14519 (2014).
9. Dassa, B., London, N., Stoddard, B. L., Schueler-furman, O. & Pietrokovski, S. Fractured genes: a novel genomic arrangement involving new split inteins and a new homing endonuclease family. Nucleic Acids Res 37, 2560-2573 (2009).
10. Carvajal-Vallejos, P., Pallisse, Roser, Mootz, H. D. & Schmidt, S. R. Unprecedented rates and efficiencies revealed for new natural split inteins from metagenomic sources. J. Biol. Chem. 287, 28686-28696 (2012).
11. Choi, J., Chen, J., Schreiber, S. L. & Clardy, J. Structure of the FKBP12-Rapamycin Complex Interacting with the Binding Domain of Human FRAP. Science 273, 239-242 (1996).
12. Aranko, a S. et al. Structure-based engineering and comparison of novel split inteins for protein ligation. Mol. Biosyst. 10, 1023-34 (2014).
13. Machleidt, T. et al. NanoBRET-A Novel BRET Platform for the Analysis of Protein-Protein Interactions. ACS Chem. Biol. 10, 1797-1804 (2015).
14. Venkatesan, K. et al. An empirical framework for binary interactome mapping. Nat. Methods 6, 83-90 (2009).
15. Lievens, S. et al. Kinase Substrate Sensor (KISS), a Mammalian In Situ Protein Interaction Sensor. Mol. Cell. Proteomics 13, 3332-3342 (2014).
16. Trepte, P. et al. LuTHy: a double-readout bioluminescence-based two-hybrid technology for quantitative mapping of protein-protein interactions in mammalian cells. Mol. Syst. Biol. 14, e8071 (2018).
17. Braun, P. et al. An experimentally derived confidence score for binary protein-protein interactions. Nat. Methods 6, 91-97 (2009).
18. Lemmon, M. a. & Schlessinger, J. Cell signaling by receptor tyrosine kinases. Cell 141, 1117-1134 (2010).
19. Zheng, Y. et al. Temporal regulation of EGF signalling networks by the scaffold protein Shc1. Nature 499, 166-71 (2013).
20. Pylayeva-Gupta, Y., Grabocka, E. & Bar-Sagi, D. RAS oncogenes: Weaving a tumorigenic web. Nat. Rev. Cancer 11, 761-774 (2011).
21. Petschnigg, J. et al. The mammalian-membrane two-hybrid assay (MaMTH) for probing membrane-protein interactions in human cells. Nat. Methods 11, 585-92 (2014).
22. Yao, Z. et al. Resource A Global Analysis of the Receptor Tyrosine Kinase-Resource A Global Analysis of the Receptor. Mol. Cell 65, 347-360 (2017).
23. Feig, L. A. & Cooper, G. M. Inhibition of NIH 3T3 Cell Proliferation by a Mutant ras Protein with Preferential Affinity for GDP. Mol. Cell. Biol. 8, 3235-3243 (1988).
24. Waas, W. F. & Dalby, K. N. Transient Protein-Protein Interactions and a Random-ordered Kinetic Mechanism for the Phosphorylation of a Transcription Factor by Extracellular-regulated Protein Kinase 2. J. Biol. Chem. 277, 12532-12540 (2002).
25. Garai, A. et al. Specificity of Linear Motifs That Bind to a Common Mitogen-Activated Protein Kinase Docking Groove. Sci. Signal. 5, ra74 (2012).
26. Floyd, B. J. et al. Mitochondrial Protein Interaction Mapping Identifies Regulators of Respiratory Chain Function Article Mitochondrial Protein Interaction Mapping Identifies Regulators of Respiratory Chain Function. Mol. Cell 63, 621-632 (2016).
27. Jackson, T. D., Palmer, C. S. & Stojanovski, D. Mitochondrial diseases caused by dysfunctional mitochondrial protein import. Biochem. Soc. Trans. 46, 1225-1238 (2018).
28. Kozjak-Pavlovic, V. The MICOS complex of human mitochondria. Cell Tissue Res. 367, 83-93 (2017).
29. Ciszak, E. M., Korotchkina, L. G., Dominiak, P. M., Sidhu, S. & Patel, M. S. Structural Basis for Flip-Flop Action of Thiamin Pyrophosphate-dependent Enzymes Revealed by Human Pyruvate Dehydrogenase. 278, 21240-21246 (2003).
30. Yaish, P., Gazit, A., Gilon, C. & Levitzki, A. Blocking of EGF-Dependent Cell Proliferation by EGF Receptor Kinase Inhibitors. Science 242, 933-935 (1988).
31. Roberts, A. W., Stilgenbauer, S., Seymour, J. F. & Huang, D. C. S. Venetoclax in Patients with Previously Treated Chronic Lymphocytic Leukemia. Clin. Cancer Res. 23, 4527-4534 (2017).
32. Souers, A. J. et al. ABT-199, a potent and selective BCL-2 inhibitor, achieves antitumor activity while sparing platelets. Nat. Med. 19, 202-208 (2013).
33. Stagljar, I. The power of OMICs. Biochem. Biophys. Res. Commun. 479, 607-609 (2016).
34. Barrios-Rodiles, M. et al. High-Throughput Mapping of a Dynamic Signaling Network in Mammalian Cells. Science 307, 1621-1625 (2005).
35. Lemmens, I. et al. Heteromeric MAPPIT: a novel strategy to study modification-dependent protein-protein interactions in mammalian cells. Nucleic Acids Res. 31, e75 (2003).
36. Fields, S. & Song, O. A Novel Genetic System to Detect Protein-Protein Interactions. Nature 340, 245-246 (1989).
37. Galarneau, A., Primeau, M., Trudeau, L. & Michnick, S. W. β-Lactamase protein fragment complementation assays as in vivo and in vitro sensors of protein-protein interactions. Nat. Biotechnol. 20, 619-622 (2002).
38. Ramachandra, N. et al. Self-Assembling Protein Microarrays. Science 305, 86-91 (2004).
39. Olhovsky, M. et al. OpenFreezer: a reagent information management software system. Nat. Methods 8, 612-613 (2011).
40. Boussif, O. et al. A versatile vector for gene and oligonucleotide transfer into cells in culture and in vivo: Polyethylenimine. Proc. Natl. Acad. Sci. U.S.A. 92, 7297-7301 (1995).
41. Kotlyar, M., Pastrello, C., Malik, Z. & Jurisica, I. IID 2018 update: Context-specific physical protein-protein interactions in human, model organisms and domesticated species. Nucleic Acids Res. 47, D581-D589 (2019).
42. Kotlyar, M. et al. In silico prediction of physical protein interactions and characterization of interactome orphans. Nat. Methods 12, 79-84 (2014).
43. Boxem, M. et al. A Protein Domain-Based Interactome Network for C. elegans Early Embryogenesis. Cell 134, 534-545 (2008).
44. Simonis, N. et al. Empirically controlled mapping of the Caenorhabditis elegans protein-protein interactome network. Nat. Methods 6, 47-54 (2009).

Unless otherwise defined, all technical and scientific terms used herein have the same meaning as commonly understood by one of ordinary skill in the art to which this invention belongs.

It should be understood that the materials, methods, and examples provided here are representative of preferred embodiments, are exemplary, and are not intended as limitations on the scope of the invention.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other embodiments are set forth within the following claims.

1. An artificial split intein, wherein the artificial split intein comprises (i) a C-terminus fragment (IC) that includes amino acid residues at positions 13 to 37 of wild type IC of GP41-1, and a N-terminus fragment (IN) that includes amino acid residues at positions 1 to 88 of the wild type IN of GP41-1 fused to amino acid residues 1 to 12 of wild type IC of GP41-1 (C25 GP41-1 split intein), or (ii) an IC that includes amino acids at positions 14 to 37 of wild type IC of GP41-1 and an IN that includes amino acid residues at positions 1 to 88 of wild type IN of GP41-1 fused to amino acid residues at positions 1 to 13 of wild type IC of GP41-1 (C24 GP41-1 split intein), or (iii) an IC that includes amino acids at positions 15 to 37 of wild type IC of GP41-1 and an IN that includes amino acid residues at positions 1 to 88 of wild type IN of GP41-1 fused to amino acid residues at positions 1 to 14 of wild type IC of GP41-1 (C23 GP41-1 split intein).

2. A system for detecting interactions between a first protein or fragment thereof (bait protein) and a second protein or fragment thereof (prey protein) comprising:
(a) a bait construct comprising the bait protein, a first epitope tag and an intein N-terminal fragment (IN); and
(b) a prey construct comprising the prey protein, a second epitope tag, and an intein C-terminal fragment (IC),
wherein (i) the IC includes amino acid residues at positions 13 to 37 of wild type IC of GP41-1, and the IN includes amino acid residues at positions 1 to 88 of the wild type IN of GP41-1 fused to amino acid residues 1 to 12 of wild type IC of GP41-1 (C25 GP41-1 split intein), or (ii) the IC includes amino acids at positions 14 to 37 of wild type IC of GP41-1 and the IN includes amino acid residues at positions 1 to 88 of wild type IN of GP41-1 fused to amino acid residues at positions 1 to 13 of wild type IC of GP41-1 (C24 GP41-1 split intein), or (iii) the IC includes amino acids at positions 15 to 37 of wild type IC of GP41-1 and the IN includes amino acid residues at positions 1 to 88 of wild type IN of GP41-1 fused to amino acid residues at positions 1 to 14 of wild type IC of GP41-1 (C23 GP41-1 split intein).

3. The system of claim 2, wherein the bait protein is fused at its C-terminus to the IN through the first epitope tag, and wherein the prey protein is fused at its N-terminus to the IC through the second epitope tag.

4. The system of claim 2, wherein the IN is fused to the N-terminal end of the bait protein while keeping the first epitope tag upstream (NIN), and the prey protein is fused at its N-terminus to the IC through the second epitope tag.

5. The system of claim 2, wherein the bait protein is fused at its C-terminus to the IN through the first epitope tag, and wherein the IC is fused to the C-terminal end of the prey protein while keeping the second epitope tag downstream (CIC).

6. The system of claim 2, wherein the bait construct further comprises a third epitope tag in tandem with the first tag.

7. The system of claim 6, wherein the first epitope tag, the second epitope tag and the third epitope tag include FLAG, V5-tag, Myc-tag, hemagglutinin (HA)-tag, Spot-tag and NE-tag.

8. (canceled)

9. The system of claim 2, wherein the bait protein is a soluble or membrane protein or fragment thereof.

10. The system of claim 2, wherein the prey protein is a soluble or membrane protein or fragment thereof.

11. A method for detecting the interaction between a first protein or part thereof (bait protein) and a second protein or part thereof (prey protein) comprising:
(a) providing a bait construct as defined in claim 2;
(b) providing a prey construct as defined in claim 2, wherein an association of the bait protein and the prey protein results in the IN and IC reconstituting into a functional intein molecule which then catalyzes its excision and formation of an intact protein which includes the first epitope tag and the second epitope tag;
(c) incubating the bait construct and the prey construct under conditions that allow the formation of the intact protein to form an incubate; and
(d) adding to the incubate a detectable agent that recognizes at least one of the first epitope tag and the second epitope tag to detect the formation of the intact protein, whereby detection of the intact protein being indicative that the first protein or part thereof and the second protein or part thereof interact.

12. The method of claim 11, wherein the detectable agent is an antibody.

13. The method of claim 11, wherein the method further comprises measuring an expression output of the detected intact protein as a measure of an amount of interaction between the first and the second proteins to quantitatively measure strength and affinity between the bait protein and the prey protein.

14. The method of claim 11, wherein the bait construct and the prey construct are expressed in a host cell.

15. The method of claim 14, wherein the bait construct and the prey construct are expressed in the host cell by:
(i) introducing into the host cell as part of a bait vector, a first gene under the control of a promoter, said first gene coding inter alia for the bait protein which gene is attached to the DNA-sequence of a first module encoding inter alia the first epitope tag and the IN; and
(ii) introducing into the host cell, as part of a prey vector, a second gene under the control of a promoter, the second gene coding inter alia for the prey protein which gene is attached to the DNA sequence of a second module encoding inter alia the second epitope tag and the IC.

16. The method of claim 15, wherein the bait vector is maintained episomally in the host cell or is integrated into the genome of the host cell.

17. The method of claim 15, wherein the prey vector is maintained episomally in the host cell or is integrated into the genome of the host cell.

18. The method of claim 12, wherein the bait construct further comprises a third epitope tag in tandem with the first epitope tag, and wherein the method further comprises performing another incubation of the incubate on a substrate coated with an anti-first epitope tag antibody for immunological immobilization of the bait construct to the substrate and wherein the detectable agent is an anti-third epitope tag antibody that allows for detection of the bait construct against either the first epitope tag or the third epitope tag.

19. The method according to claim 18, wherein the detection is performed as an ELISA assay or as a Homogeneous Time Resolved Fluorescence (HTRF) assay.

20. A method of identifying a potentially pharmaceutically active inhibitor of an interaction between a bait protein and a prey protein comprising:
(a) providing a host cell;
(b) expressing in the host cell the system of claim 2, the bait protein and the prey protein being selected such that they interact when expressed in the host cell, wherein the interaction of the bait protein and the prey protein results in the IN and IC reconstituting into a functional intein molecule which then catalyzes its excision and formation of an intact protein which includes the first epitope tag and the second epitope tag;
(c) incubating the host cell in presence of a compound of interest under conditions that allow for the formation of the intact protein to form an incubate; and
(e) adding to the incubate an antibody or antibodies that recognize at least one or both of the first epitope tag and the second epitope tag to detect the formation of the intact protein, wherein an absence of detection of the intact protein is indicative of the compound of interest being potentially pharmaceutically active inhibitor of the interaction between the prey protein and the bait protein.

21. A method for providing a compound that can interfere with protein/protein interaction, the method comprising: (a) providing host cells having the system of claim 2, the bait protein and the prey protein being selected such that they interact when expressed in the host cells to form an intact protein; (b) incubating a first set of the host cells under conditions that allow for expression of the intact protein in the presence of a compound of interest to obtain a first incubation and incubating a second set of the host cells under conditions that allow for expression of the intact protein in the absence of the compound of interest to obtain a second incubation; (c) measuring a level of expression of the intact protein in the first incubation and a level of expression of the intact protein in the second incubation, wherein when the level of expression of the intact protein in the first incubation is lower than the level of expression of the intact protein in the second incubation indicates that the compound interest interferes with protein/protein interaction, thereby providing the compound that can interfere with protein/protein interaction.

22. An isolated peptide comprising SEQ ID NO:3.

23. An isolated peptide comprising SEQ ID NO:4.
